(19) United States
(12) Patent Application Publication
(10) Pub. No.: US 2024/0428884 A1
(43) Pub. Date: Dec. 26, 2024

(54) Analyzing and Merging Data from Genome-Wide Association Studies

(71) Applicant: 23andMe, Inc., Sunnyvale, CA (US)

(72) Inventors: Karl A. Heilbron, Sunnyvale, CA (US); Xin Wang, San Francisco, CA (US); Shubham Saini, San Jose, CA (US); Yanyu Liang, Mountain View, CA (US); Qiaojuan Jane Su, San Jose, CA (US); Robert K. Bell, Menlo Park, CA (US); Alan M. Kwong, Los Angeles, CA (US); Andrew O. Stiles, Sunnyvale, CA (US)

(21) Appl. No.: 18/736,239

(22) Filed: Jun. 6, 2024

Related U.S. Application Data
(60) Provisional application No. 63/509,644, filed on Jun. 22, 2023.

(51) Int. Cl. G16B 20/20

(52) U.S. Cl. CPC G16B 20/20

(57) ABSTRACT

Example embodiments relate to analyzing and merging data from genome-wide association studies. An example embodiment includes a method. The method includes receiving, by a processor from a memory, a candidate data set including data from a genetic study conducted within a population. The data from the genetic study includes a plurality of gene variants determined within the population. The method also includes removing one or more of the plurality of gene variants from the candidate data set in order to generate a revised candidate data set based on one or more variant-level quality metrics. Further, the method includes determining whether the revised candidate data set satisfies one or more study-level quality metrics. Additionally, the method includes establishing data set metadata based on whether the revised candidate data set satisfies one or more study-level quality metrics. Further, the method includes storing, within the memory, the data set metadata.

### CROSS-REFERENCE TO RELATED APPLICATIONS

The present application claims the benefit of U.S. Provisional Application No. 63/509,644, filed Jun. 22, 2023. The contents of which are hereby incorporated by reference in their entirety.

### BACKGROUND

Genome-wide association studies (GWASs) are research projects performed to analyze genetic variants across populations of individuals. In many cases, GWASs may be performed to establish relationships between phenotypes (e.g., diseases in humans) and gene variants (e.g., single-nucleotide polymorphisms (SNPs)) across a population. Such relationships may be established based on reported or measured phenotypes for individuals within the population along with genetic sequences of the individuals within the population. If a certain gene variant has a statistically significant correlation with a certain phenotype (e.g., a disease), that gene variant may be deemed to be associated with that phenotype (e.g., might indicate an individual carrying the risk variant having an increased risk for that disease).

### SUMMARY

Example embodiments described herein include techniques for merging and/or analyzing data from different genetic studies (e.g., different GWASs). In order to perform such merging and/or analyzing, the disclosed techniques may include performing one or more quality control techniques on a genetic study to ensure that it is sufficient quality to warrant inclusion in a combined data set. Such quality control techniques may include applying one or more variant-level quality metrics and/or study-level quality metrics to the genetic study under consideration. Further, in order to analyze data from different genetic studies, phenotypes provided in one genetic study may be analogized to phenotypes provided in another genetic study (e.g., in order to determine whether, despite separate underlying nomenclatures, phenotypes from the different genetic studies sufficiently overlap such that they can be considered related and/or identical). Additionally, prior to performing an analysis on data from different genetic studies, the relative sizes between the different genetic studies may be compared (e.g., to determine whether each of the genetic studies is large enough to non-negligibly contribute to a data set made up of the genetic studies combined).

In a first aspect, a method is provided. The method includes receiving, by a processor from a memory, a candidate data set that includes data from a genetic study conducted within a population. The data from the genetic study includes a plurality of gene variants determined within the population. The method also includes removing, by the processor, one or more of the plurality of gene variants from the candidate data set in order to generate a revised candidate data set based on one or more variant-level quality metrics. Additionally, the method includes determining, by the processor, whether the revised candidate data set satisfies one or more study-level quality metrics. Further, the method includes establishing, by the processor, data set metadata based on whether the revised candidate data set satisfies one or more study-level quality metrics. In addition, the method includes storing, by the processor within the memory, the data set metadata.

In a second aspect, a method is provided. The method includes receiving, by a processor, a first data set including genetic data associated with a first population. The genetic data includes a plurality of first phenotypes associated with a plurality of first gene variants determined within the first population. The method also includes receiving, by the processor, a second data set including data from a genetic study conducted within a second population. The data from the genetic study includes a plurality of second phenotypes associated with a plurality of second gene variants determined within the second population. Additionally, the method includes, for each of the first phenotypes, determining, by the processor for each of the second phenotypes, a similarity score between the respective first phenotype and the respective second phenotype. Determining the similarity score includes comparing the first phenotype to the second phenotype using an ontology. Further, the method includes, for each of the first phenotypes, comparing, by the processor, each of the respective similarity scores to a threshold similarity score. In addition, the method includes, for each of the first phenotypes, adding, by the processor for each of the respective similarity scores that is greater than the threshold similarity score, the first phenotype and the second phenotype associated with the respective similarity score to a set of pairs of related phenotypes when the first phenotype and the second phenotype associated with the respective similarity score are both case-control phenotypes or both continuous phenotypes. Still further, the method includes determining, by the processor, a ratio of the second population size to the first population size. Even further, the method includes comparing, by the processor, the ratio to a threshold ratio. Yet further, the method includes performing, by the processor, additional analysis on the set of pairs of related phenotypes when the ratio is greater than the threshold ratio.

In a third aspect, a non-transitory, computer-readable medium having instructions stored thereon is provided. The instructions, when executed by a processor, cause the processor to receive, from a memory, a candidate data set including data from a genetic study conducted within a population. The data from the genetic study includes a plurality of gene variants determined within the population. The instructions, when executed by the processor, also cause the processor to remove one or more of the plurality of gene variants from the candidate data set in order to generate a revised candidate data set based on one or more variant-level quality metrics. Additionally, the instructions, when executed by the processor, cause the processor to determine whether the revised candidate data set satisfies one or more study-level quality metrics. Further, the instructions, when executed by the processor, cause the processor to establish data set metadata based on whether the revised candidate data set satisfies one or more study-level quality metrics. In addition, the instructions, when executed by the processor, cause the processor to store, within the memory, the data set metadata.

In a fourth aspect, a non-transitory, computer-readable medium having instructions stored thereon is provided. The instructions, when executed by a processor, cause the processor to receive a first data set including genetic data associated with a first population. The genetic data includes a plurality of first phenotypes associated with a plurality of first gene variants determined within the first population. The instructions, when executed by the processor, also cause the processor to receive a second data set including data from a genetic study conducted within a second population. The data from the genetic study includes a plurality of second phenotypes associated with a plurality of second gene variants determined within the second population. Additionally, the instructions, when executed by the processor, cause the processor to, for each of the first phenotypes, determine, for each of the second phenotypes, a similarity score between the respective first phenotype and the respective second phenotype. Determining the similarity score includes comparing the first phenotype to the second phenotype using an ontology. Further, the instructions, when executed by the processor, cause the processor to, for each of the first phenotypes, compare each of the respective similarity scores to a threshold similarity score. In addition, the instructions, when executed by the processor, cause the processor to, for each of the first phenotypes, add, for each of the respective similarity scores that is greater than the threshold similarity score, the first phenotype and the second phenotype associated with the respective similarity score to a set of pairs of related phenotypes when the first phenotype and the second phenotype associated with the respective similarity score are both case-control phenotypes or both continuous phenotypes. Still further, the instructions, when executed by the processor, cause the processor to determine a ratio of the second population size to the first population size. Yet further, the instructions, when executed by the processor, cause the processor to compare the ratio to a threshold ratio. Even further, the instructions, when executed by the processor, cause the processor to perform additional analysis on the set of pairs of related phenotypes when the ratio is greater than the threshold ratio.

In a fifth aspect, a system is provided. The system includes one or more processors. The one or more processors are configured to receive, from a memory, a candidate data set including data from a genetic study conducted within a population. The data from the genetic study includes a plurality of gene variants determined within the population. The one or more processors are also configured to remove one or more of the plurality of gene variants from the candidate data set in order to generate a revised candidate data set based on one or more variant-level quality metrics. Additionally, the one or more processors are configured to determine whether the revised candidate data set satisfies one or more study-level quality metrics. Further, the one or more processors are configured to establish data set metadata based on whether the revised candidate data set satisfies one or more study-level quality metrics. In addition, the one or more processors are configured to store, within the memory, the data set metadata.

In a sixth aspect, a system is provided. The system includes one or more processors configured to receive a first data set including genetic data associated with a first population. The genetic data includes a plurality of first phenotypes associated with a plurality of first gene variants determined within the first population. The one or more processors are also configured to receive a second data set including data from a genetic study conducted within a second population. The data from the genetic study includes a plurality of second phenotypes associated with a plurality of second gene variants determined within the second population. Additionally, the one or more processors are configured to, for each of the first phenotypes, determine, for each of the second phenotypes, a similarity score between the respective first phenotype and the respective second phenotype. Determining the similarity score includes comparing the first phenotype to the second phenotype using an ontology. Further, the one or more processors are configured to, for each of the first phenotypes, compare each of the respective similarity scores to a threshold similarity score. In addition, the one or more processors are configured to, for each of the first phenotypes, add, for each of the respective similarity scores that is greater than the threshold similarity score, the first phenotype and the second phenotype associated with the respective similarity score to a set of pairs of related phenotypes when the first phenotype and the second phenotype associated with the respective similarity score are both case-control phenotypes or both continuous phenotypes. Still further, the one or more processors are configured to determine a ratio of the second population size to the first population size. Even further, the one or more processors are configured to compare the ratio to a threshold ratio. Yet further, the one or more processors are configured to perform additional analysis on the set of pairs of related phenotypes when the ratio is greater than the threshold ratio.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

FIG. 1 is a block diagram illustrating a computing system, according to example embodiments.

FIG. 2 illustrates a network, according to example embodiments.

FIG. 3A is a map illustrating genetic studies performed in different locations around the world.

FIG. 3B is an illustration of a genetic study, according to example embodiments.

FIG. 3C is an illustration of a genetic study, according to example embodiments.

FIG. 4A is a communication flow diagram of a network, according to example embodiments.

FIG. 4B is an illustration of a quality assessment, according to example embodiments.

FIG. 4C is a communication flow diagram of a network, according to example embodiments.

FIG. 5A is an illustration of a list of gene variants in a candidate data set, according to example embodiments.

FIG. 5B is an illustration of an application of variant-level quality metrics, according to example embodiments.

FIG. 5C is an illustration of a list of gene variants in a revised candidate data set, according to example embodiments.

FIG. 5D is an illustration of a list of genetic studies, according to example embodiments.

FIG. 5E is an illustration of an application of study-level quality metrics, according to example embodiments.

FIG. 5F is an illustration of a list of genetic studies with associated metadata based on the application of study-level quality metrics, according to example embodiments.

FIG. 6 is a communication flow diagram of a network, according to example embodiments.

FIG. 7 is an illustration of a plurality of phenotypes arranged according to an ontology, according to example embodiments.

FIG. 8 is a flowchart illustration of a method, according to example embodiments.

FIG. 9 is a flowchart illustration of a method, according to example embodiments.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. In addition, some of the illustrated elements may be combined or omitted. Similarly, an example embodiment may include elements that are not illustrated in the figures.

### I. OVERVIEW

The following description and accompanying drawings will elucidate features of various example embodiments. The embodiments provided are by way of example, and are not intended to be limiting. As such, the dimensions of the drawings are not necessarily to scale.

GWASs may have relatively small sample sizes and/or may only capture certain classes of phenotypes. Additionally, GWASs tend to be performed by specific research groups, using specific classification techniques, and/or only on populations from a specific geographic region, of a specific biological sex, of a specific race, etc. Hence, it would be beneficial to be able to combine the results from multiple GWASs to perform larger scale studies. As such, example embodiments herein provide techniques for statistically combining two or more GWASs in order to provide more robust data across a larger sample size. For example, results from a first GWAS (aggregating statistical analysis of the individuals forming cases and controls in the first GWAS) may be statistically combined with results from a second GWAS (aggregating statistical analysis of the individuals forming cases and controls in the second GWAS) to yield a meta-analysis that has a larger sample size and statistical power than the first and second GWAS by themselves.. Such a meta-analysis may include, for example, assessing whether a GWAS signal from the first GWAS colocalizes (e.g., shares a causal variant) with a GWAS signal for a different phenotype from the second GWAS. Further, in some embodiments, the meta-analysis of the first GWAS and the second GWAS may be additionally meta-analyzed with yet another GWAS (e.g., a third GWAS). Such an additional meta-analysis could be used to test for a genetic correlation in the third GWAS based on the same phenotype analyzed in the first GWAS or the second GWAS or based on a different phenotype. While GWAS data is used throughout this disclosure as an example, it is understood that the techniques described herein could also be used to evaluate and combine other forms of genetic data related to various populations.

Prior to performing the meta-analysis (or other statistical analysis) on the GWASs, some compatibility issues may arise. Hence, embodiments described herein may be used to identify GWAS(s) from among one or more candidate GWASs that are of sufficient quality to be used in a meta-analysis of the candidate GWASs (referred to herein as performing a “quality assessment”). Further, embodiments herein may include techniques that statistically combine two or more GWASs or other genetic data in a downstream meta-analysis, colocalization, or other statistical analysis (referred to herein as performing a “merging technique”). In various examples, two or more GWASs may define substantially similar phenotypes in different ways. Hence, embodiments disclosed herein include techniques for comparing the various phenotypes of multiple GWASs (e.g., GWASs that use different naming conventions) such that a unified set of phenotypes can be provided in the meta-analysis. This allows for additional meaningful determinations to be made on the combined population within the statistically combined GWASs.

Various techniques described herein provide improvements to a specific technology or technological field (e.g., to the field of epidemiology, to the study of pathogenic gene variants, and/or to the field of population genetics). In particular, as described above, the ability to perform studies that combine information from multiple previously generated studies (e.g., previously generated GWASs) can enhance the ability to identify genetic factors for underlying phenotypes/diseases (e.g., by meta-analyzing a larger sample size and/or individuals from different populations) and/or provide information on the interplay between multiple phenotypes/diseases and/or multiple gene variants (e.g., by meta-analyzing multiple underlying data sets where the underlying data sets include studies of different phenotypes/diseases from one another). Without the techniques described herein, a meta-analysis that includes meaningful statistical inferences could not be produced when considering multiple GWASs where disparate phenotype labeling techniques and/or gene variant labeling techniques have been used. As such, in the absence of the techniques described herein, either: meta-analysis from which additional statistical understanding can be drawn will not be performed (e.g., thereby impeding enhanced understanding of genetic sources of disease, illness, aging, etc.) or significant additional effort will need to be performed in advance of such analysis (e.g., a laborious complete relabeling of data within one or more of the genetic studies or a complete regathering of the data from all of the underlying data sets such that all of the data incorporates the same labeling from the beginning).

Additionally, embodiments described herein provide techniques that improve the functioning of a computer. In some embodiments, the quality assessments and merging techniques described herein can reduce computation time and/or memory requirements. For example, when two or more data sets are meta-analyzed, some redundant data may be eliminated and/or, at the very least, may be identified such that unnecessary analysis does not occur in the future. For example, phenotype descriptions that overlap between the underlying data sets may be identified and de-duplicated. Additionally or alternatively, phenotypes from different underlying data sets that are sufficiently similar may be stored within a set of pairs of related phenotypes. Thereafter, when a meta-analysis is being performed, certain calculations need not be duplicated. For example, when a calculation is performed for one phenotype, it may be assumed that the calculation equally applies for a phenotype paired with that phenotype within the set of phenotype pairs such that the calculation need not be performed again for the paired phenotype. Likewise, the value associated with a result of such a calculation need only be stored once (e.g., since that same value is known to be relevant to multiple phenotypes). In addition, if it is determined that the information provided by one of the data sets is of relatively incremental or negligible value, the meta-analysis may not be performed (e.g., further saving computing resources).

Further, as described herein, the quality assessments according to example embodiments may conserve computing resources (e.g., memory, processing power, and/or computation time), as well. For example, quality assessments may include identifying entire candidate data sets that should not be retained in memory and/or meta-analyzed with another data set because they do not satisfy one or more study-level quality metrics. Similarly, quality assessments may also include eliminating certain gene variants from candidate data sets when those gene variants do not meet certain variant-level quality metrics. In this way, the amount of data that is stored within a revised candidate data set (e.g., and, perhaps ultimately, is meta-analyzed with another underlying data set) may be reduced while maintaining the statistically valuable data. Further, these quality assessments can prevent future computation time from being spent analyzing studies and/or variants that do not include data of sufficient quality to warrant analysis. Given the above examples, as well as others contemplated herein, it is understood that example embodiments provide improvements to computer functionality as well as to computer-related technology.

Example quality assessment techniques described herein may include evaluating a candidate data set associated with a GWAS to determine whether that candidate data set is of sufficient quality (e.g., of sufficient quality to be included in an eventual meta-analysis of the GWASs or other statistical combination). To make such a determination, a number of variant-level quality metrics may be applied to the candidate data set. If any of the gene variants in the candidate data set fail to satisfy the variant-level quality metrics, those gene variants may be removed from the candidate data set (e.g., which may result in those gene variants not being included in an eventual downstream statistical analysis of the GWASs).

Variant-level quality metrics may include such metrics as a minimum threshold number of occurrences of a given gene variant within the GWAS (e.g., to eliminate exceedingly rare/statistically insignificant gene variants). The minimum threshold number of occurrences may be determined based on an alternative data set from an alternative GWAS (e.g., where the alternative data set is ultimately to be meta-analyzed with the candidate data set). For example, a multiple (e.g., two times, three times, four times, five times, etc.) of a minor allele frequency for an analogous gene variant within the alternative data set may be used as the minimum threshold number of occurrences. In addition to or instead of ensuring a minimum threshold number of occurrences for a given gene variant, variant-level quality metrics may also be used to eliminate duplicate gene variants within the candidate data set, gene variants within the candidate data set that are missing pieces of information (e.g., information defined by a variant taxonomy, such as a chromosome, position, reference allele, and alternative allele (CPRA) identification), gene variants within the candidate data set that are on the Y chromosome, gene variants within the candidate data set that have incomplete variant metadata, gene variants within the candidate data set that are identical to other gene variants in the candidate data set, gene variants within the candidate data set that have associated p-values less than 0 or greater than 1, and/or gene variants within the candidate data set that have associated error of less than or equal to 0. Other variant-level quality metrics are also possible and are contemplated herein.

After removing any gene variants that do not satisfy the variant-level quality metrics, a revised candidate data set will result. This revised candidate data set may then be reviewed to determine whether it satisfies a number of study-level quality metrics. If the revised candidate data set satisfies each of the study-level quality metrics, the revised candidate data set may then be stored (e.g., within a memory, such as a cloud storage) for later access. Additionally or alternatively, data set metadata (e.g., indications of pass or fail relative to one or more of the study-level quality metrics, indications of pass or fail relative to one or more of the variant-level quality metrics, one or more scores determined while applying the study-level quality metrics, one or more scores determined while applying the variant-level quality metrics, etc.) may be established based on the revised candidate data set. This data set metadata may be associated with the candidate data set and stored along with the candidate data set for later retrieval. For example, a query requesting only candidate data sets which pass a given study-level quality metric may be performed. The data set metadata associated with each of the potential candidate data sets within a data storage (e.g., within cloud storage) may be reviewed to identify only those candidate data sets which satisfy the given study-level quality metric (e.g., without necessarily reapplying the variant-level and/or study-level quality metrics to the candidate data sets).

In some embodiments, after determining that a revised candidate data set satisfies each of the study-level quality metrics, the revised candidate data set may be merged with another data set (e.g., another revised data set that was revised based on variant-level quality metrics and study-level quality metrics) to perform a meta-analysis of the GWASs. Example study-level quality metrics employed according to the techniques described herein may include a minimum threshold number of unique gene variants (e.g., 1,000, 10,000, 100,000, 1 million, or 10 million) within the revised candidate data set, a minimum population size within the revised candidate data set, a maximum threshold proportion (e.g., 0.01, 0.05, or 0.1) of gene variants within the revised candidate data set that either are identical to other gene variants within the revised candidate data set or are missing one or more pieces of information (e.g., defined by an associated variant taxonomy), whether the data in the revised candidate data set is whole-exome data, or threshold values related to statistics (e.g., associated with odds ratios or associated with one or more chi-squared tests) for the collection of gene variants within the revised candidate data set. Other study-level quality metrics are also possible and are contemplated herein.

Example merging techniques described herein may be performed to merge two or more data sets (e.g., from two or more separate GWASs) to perform a meta-analysis or any other statistical combination of multiple datasets described herein. For example, a merging technique may include combining a first data set that includes genetic data associated with a first population with a second data set that includes data from a GWAS conducted within a second population. The first data set may include a plurality of phenotypes associated with a plurality of gene variants determined for the first population. Likewise, the second data set may include a plurality of phenotypes associated with a plurality of gene variants determined for the second population. In order to meaningfully combine the first data set with the second data set, each of the phenotypes listed in the second data set may be compared to each of the phenotypes listed in the first data set (e.g., using one or more lists of published tags and associated descriptions and/or using mapping techniques applied from a most-effective mapping technique to a least-effective mapping technique until a tag is determined for a given phenotype). The comparisons can include multiple stages. For example, a first stage may include mapping phenotypes of a first data set and/or phenotypes of a second data set to various ontology tags (e.g., using term frequency-inverse document frequency (TF-IDF) and edit distance). A second stage may include leveraging the structure of the ontology to determine similarity. In some embodiments, for example, information theory can be used to determine similarity. These comparisons may each yield a similarity score. Further, the similarity score may be determined using an ontology (e.g., the Medical Subject Headings (MeSH) ontology or the Experimental Factor Ontology (EFO)). Each of these similarity scores may then be compared to a threshold similarity score (e.g., 0.75, 0.8, 0.85, 0.9, 0.95, 0.99, or 0.999). If a respective similarity score is greater than the threshold similarity score, the respective phenotypes being compared from the first data set and the second data set may be determined to be related. Further, for any related phenotypes from the two data sets that are both case-control phenotypes or both continuous phenotypes (i.e., for related phenotypes that are not of dissimilar type when it comes to case-control vs. continuous), those related phenotypes may be added to a set of pairs of related phenotypes. When the combination of the first data set and the second data set is completed, the combined data set may include (e.g., as data set metadata) the set of pairs of related phenotypes (e.g., listing all phenotypes within the combined dataset that are determined to be similar). This set of pairs of related phenotypes can be used downstream (e.g., when performing a statistical analysis on the GWASs, such as a meta-analysis).

In addition, when adding a second data set to a first data set to form a combined data set, the relative sizes of the data sets may be compared. For example, a ratio between the sample size of the second data set and the sample size of the first data set may be determined. This ratio may then be compared to a threshold ratio (e.g., to ensure that the contribution by the second data set to the combined data set is meaningful, in a relative sense). For example, if the first data set is an order of magnitude (or even 2, 3, 4, 5, 6, etc. orders of magnitude) larger than the second data set, there might not be significant value added by incorporating the second data set to form a combined data set. In such cases where the ratio is not greater than the threshold ratio, a determination may be made that additional analysis (e.g., a meta-analysis on the GWASs) is unwarranted (i.e., sufficiently valuable additional information may not be rendered by performing additional analysis on the combined data set). Hence, additional analysis may only be performed in cases where the relative contribution of the second data is non-negligible.

The additional statistical analyses (i.e., meta-analyses) that can be performed are widely varied. For example, descriptions of various phenotypes could be compared (e.g., potentially leading to still further analysis if it turns out that previously unidentified relationships between gene variants are present based on corresponding phenotypes). Such descriptions may be stored as phenotype metadata associated with the various phenotypes. Further, comparing the descriptions of various phenotypes may include determining whether such descriptions share a threshold extent of similarity and/or whether a description of one phenotype represents a subset or a superset of a description of another phenotype.

In some embodiments, the additional analyses may involve determining how many individuals within the combined data set are unique (i.e., establishing whether any individual is included two or more times in the combined data set because that individual was present in two or more of the underlying data sets that were combined to form the combined data set). The number of unique individuals may provide an accurate count of the population size considered in the combined data set. Further, individuals that are included more than once may be accounted for when calculating statistics associated with phenotypes and/or gene variants within the combined data set.

Further, the additional analyses may include analyzing variants within a locus to identify whether multiple variants are associated independently with a phenotype. Determining whether multiple variants within the locus are independently associated with the phenotype may include applying conditional and joint association analysis using data set metadata associated with the second data set (e.g., including summary statistics and/or a linkage disequilibrium reference panel). If the locus does include multiple variants independently associated with the phenotype, this may provide information regarding each variants' effects on the underlying phenotype.

### II. EXAMPLE SYSTEMS

The following description and accompanying drawings will elucidate features of various example embodiments. The embodiments provided are by way of example, and are not intended to be limiting. As such, the dimensions of the drawings are not necessarily to scale.

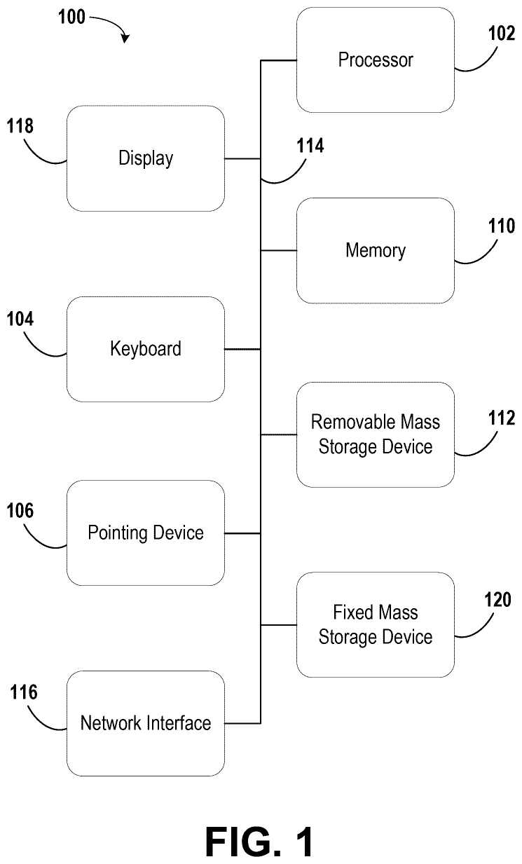

FIG. 1 is a functional diagram illustrating a programmed computing system for performing the techniques described herein (e.g., the quality assessment and the merging techniques disclosed herein), according to example embodiments. Computing system 100, which includes various subsystems as described below, includes at least one microprocessor subsystem (also referred to as a processor or a central processing unit (CPU)) 102. For example, processor 102 can be implemented by a single-chip processor or by multiple processors disposed upon one or more chips. In some embodiments, processor 102 is a general purpose digital processor that controls the operation of the computing system 100. Using instructions retrieved from memory 110, the processor 102 controls the reception and manipulation of input data, and the output and display of data on output devices (e.g., display 118). In some embodiments, processor 102 includes and/or is used to provide phasing, local classification, error correction, recalibration, and/or label clustering as described below.

Processor 102 is coupled bi-directionally with memory 110, which can include a first primary storage, typically a random access memory (RAM), and a second primary storage area, typically a read-only memory (ROM). Primary storage can be used as a general storage area and as scratch-pad memo, and can also be used to store input data and processed data. Primary storage can also store programming instructions and data, in the form of data objects and text objects, in addition to other data and instructions for processes operating on processor 102. Also, primary storage typically includes basic operating instructions, program code, data, and objects used by the processor 102 to perform its functions (e.g., programmed instructions). For example, memory 110 can include any suitable computer-readable storage media, described below, depending on whether, for example, data access needs to be bi-directional or unidirectional. For example, processor 102 can also directly and very rapidly retrieve and store frequently needed data in a cache memory (not shown).

A removable mass storage device 112 provides additional data storage capacity for the computing system 100, and is coupled either bi-directionally (read/write) or unidirectionally (read only) to processor 102. For example, removable mass storage device 112 can also include computer-readable media such as magnetic tape, flash memory, PC-CARDS, portable mass storage devices, holographic storage devices, and other storage devices. A fixed mass storage device 120 can also, for example, provide additional data storage capacity. The most common example of a fixed mass storage device 120 is a hard disk drive. Mass storage devices 112, 120 generally store additional programming instructions, data, and the like that typically are not in active use by the processor 102. It will be appreciated that the information retained within mass storage devices 112, 120 can be incorporated, if needed, in standard fashion as part of memory 110 (e.g., RAM) as virtual memory.

In addition to providing processor 102 access to storage subsystems, bus 114 can also be used to provide access to other subsystems and devices. As shown, these can include a display 118, a network interface 116, a keyboard 104, and a pointing device 106, as well as an auxiliary input/output device interface, a sound card, speakers, and other subsystems as needed. For example, the pointing device 106 can be a mouse, stylus, track ball, or tablet, and is useful for interacting with a graphical user interface.

The network interface 116 allows processor 102 to be coupled to another computer, computing network, or telecommunications network using a network connection as shown. For example, through the network interface 116, the processor 102 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 102 can be used to connect the computing system 100 to an external network and transfer data according to standard protocols. For example, various process embodiments disclosed herein can be executed on processor 102, or can be performed across a network such as the Internet, intranet networks, or local area networks, in conjunction with a remote processor that shares a portion of the processing. Additional mass storage devices (not shown) can also be connected to processor 102 through network interface 116.

An auxiliary I/O device interface (not shown) can be used in conjunction with computing system 100. The auxiliary I/O device interface can include general and customized interfaces that allow the processor 102 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to storage products with a computer-readable medium (e.g., a non-transitory, computer-readable medium) that includes program code for performing various computer-implemented operations. The computer-readable medium is any data storage device that can store data which can thereafter be read by a computing system. Examples of computer-readable media include, but are not limited to, all the media mentioned above: magnetic media (e.g., hard disks, floppy disks, and magnetic tape), optical media (e.g., a compact-disk read-only memory (CD-ROM)), magneto-optical media (e.g., optical disks), and specially configured hardware devices (e.g., application-specific integrated circuits (ASICs), programmable logic devices (PLDs), and ROM and RAM devices). Examples of program code include both machine code, as produced, for example, by a compiler, or files containing higher level code (e.g., script) that can be executed using an interpreter.

The computing system shown in FIG. 1 is but an example of a computing system suitable for use with the various embodiments disclosed herein. Other computing systems suitable for such use can include additional or fewer subsystems. In addition, bus 114 is illustrative of any interconnection scheme serving to link the subsystems. Other computing architectures having different configurations of subsystems can also be utilized.

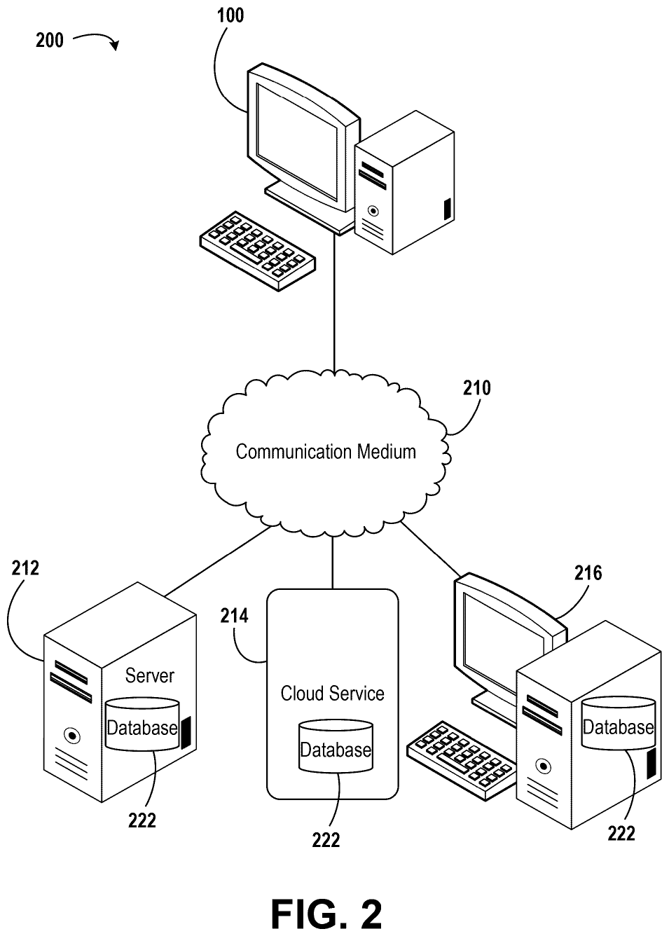

FIG. 2 illustrates a computing network 200, according to example embodiments. The computing network 200 may include a computing system (e.g., the computing system 100 shown and described with reference to FIG. 1). The computing network 200 may also include a server 212, a cloud service 214, and/or another computing system 216. Each of the server 212, the cloud service 214, and the computing system 216 may also include one or more databases 222, as illustrated. The computing system 100 may be communicatively coupled to other components of the computing network (e.g., the server 212, the cloud service 214, and/or the other computing system 216) via a communication medium 210. Additionally, one or more of the other components (e.g., the server 212, the cloud service 214, and/or the other computing system 216) may be interconnected with one another. It is understood that the computing network 200 of FIG. 2 is provided solely as an example and that other examples are also possible and are contemplated herein. For example, some computing networks according to example embodiments may include additional servers, cloud services, and/or computing systems than illustrated in FIG. 2. Alternatively, in some embodiments, the server 212, the cloud service 214, and/or the computing system 216 may be omitted from a computing network configured to perform the techniques described herein. For example, a computing network may perform the techniques described herein simply based on communications between the cloud service 214 and the computing system 100 over the communication medium 210 (e.g., without the server 212 or the computing system 216 present).

In some embodiments, the computing system 100 may include multiple components (e.g., internal computing components), as illustrated in FIG. 1. Additional or alternative components to those illustrated in FIG. 1 are also contemplated herein. Likewise, the server 212, the cloud service 214, and/or the other computing system 216 may also include one or more computing components (e.g., the same or different computing components than the computing system 100). As illustrated, the computing system 100 may correspond to a terminal device (e.g., a personal computer). Other example terminal devices are also possible and contemplated herein. For example, the computing system 100 could also include a laptop computing device, a tablet computing device, a mobile computing device, a rack-mounted server device, etc.

The server 212 may correspond to an Internet-based computing system used to store and/or process data. For example, the computing system 100 may transmit information to the server 212 via the communication medium 210 so that the server 212 may store the data for later access (e.g., for data redundancy in case the local copy on the computing system 100 is destroyed, lost, or corrupted). Additionally or alternatively, the computing system 100 may transmit data to the server 212 so that the server 212 can process the data (e.g., can perform operations on the data and/or make determinations based on the data).

The cloud service 214 may be a subscription service associated with one or more cloud servers (e.g., remote servers other than the server 212). For example, the cloud service 214 may include instructions stored within memories of multiple cloud servers and executed by processors of the multiple cloud servers. Such instructions may, when executed, allow devices (e.g., the computing system 100) to communicate with the cloud servers to store data in and retrieve data from the cloud servers. In some embodiments, the computing system 100 may have credentials (e.g., a user identification, ID, as well as an associated password) used to authenticate the computing system 100 within the cloud service 214. In various embodiments, the cloud service 214 may be located on a public cloud or a private cloud. For example, in some embodiments, the cloud service 214 may be implemented using MICROSOFT AZURE, CITRIX XENSERVER, or AMAZON WEB SERVICES CLOUD, or GOOGLE CLOUD.

The database(s) 222 may be stored within one or more memory of the respective components. For example, the database 222 of the server 212 may be stored in a non-volatile memory of the server (e.g., a hard drive). The database(s) 222 may include information used by the respective devices illustrated. For example, the database 222 of the server 212 may include information used by the server 212 to execute one or more processes of the server 212. Alternatively, the database(s) 222 may serve as a repository of information (e.g., as a cloud storage in the case of the cloud service 214) for later use by one or more other devices in the computing network 200. For example, the database 222 associated with the cloud service 214 may store (e.g., for redundancy and/or later access) information generated by/used by the computing system 100.

In some embodiments, for example, the communication medium 210 may include one or more of the following: the public Internet, a wide-area network (WAN), a local area network (LAN), a wired network (e.g., implemented using Ethernet), and a wireless network (e.g., implemented using WIFI). In order to communicate over the communication medium 210, one or more of the components in the computing network 200 may use one or more communication protocols, such as Transmission Control Protocol/Internet Protocol (TCP/IP) or User Datagram Protocol/Internet Protocol (UDP/IP).

In some embodiments, the techniques described herein regarding analyzing candidate data sets that include genetic data may include communications between two or more of the computing system 100, the server 212, the cloud service 214, and the computing system 216 illustrated in FIG. 2. For example, the server 212 may include a candidate data set (e.g., stored within the database 222). That candidate data set may be transmitted to the computing system 100 via the communication medium 210 and received by the computing system 100 at the network interface 116. After receiving the candidate data set, the computing system 100 (e.g., a processor of the computing system 100) may perform one or more calculations to evaluate the quality of the candidate data set. Based upon the quality evaluation (and potentially after one or more revisions to the candidate data set), the revised candidate data set and/or metadata associated with the revised candidate data set may be stored within the computing system 100 (e.g., within the memory 110, the removable mass storage device 112, and/or the fixed mass storage device 120) and/or transmitted to one or more other devices in the computing network 200 for storage (e.g., transmitted to the server 212 and/or the cloud service 214 over the communication medium 210 for storage in their respective databases 222).

Similarly, the techniques described herein regarding merging (e.g., and subsequently meta-analyzing) of two data sets that include genetic data may include communications between two or more of the computing system 100, the server 212, the cloud service 214, and the computing system 216 illustrated in FIG. 2. For example, the server 212 may include a first data set (e.g., stored in its database 222) and the cloud service 214 may include a second data set (e.g., stored in its database 222). The computing system 100 may receive the first data set from the server 212 and the second data set from the cloud service 214 via the communication medium 210 (e.g., at the network interface 116 of the computing system 100). The computing system 100 (e.g., the processor 102 of the computing system 100) may then relate phenotypes within the first data set to phenotypes within the second data set (e.g., using an ontology) and/or make comparisons (e.g., about relative population size) between the first data set and the second data set. Based on the outcome of such relationships and/or comparisons, the computing system 100 (e.g., the processor 102 of the computing system 100) may determine whether additional analysis on related phenotypes within the two (or more) data sets is warranted and/or perform such additional analyses. The determined relationships, the results of the comparisons, and/or the results of the additional relationships may be stored within the computing system 100 (e.g., the memory 110, the removable mass storage device 112, and/or the fixed mass storage device 120). Additionally or alternatively, the determined relationships, the results of the comparisons, and/or the results of the additional relationships may be transmitted by the computing system 100 to the server 212, the cloud service 214 and/or the computing system 216 (e.g., for storage in their respective databases 222).

### III. EXAMPLE PROCESSES

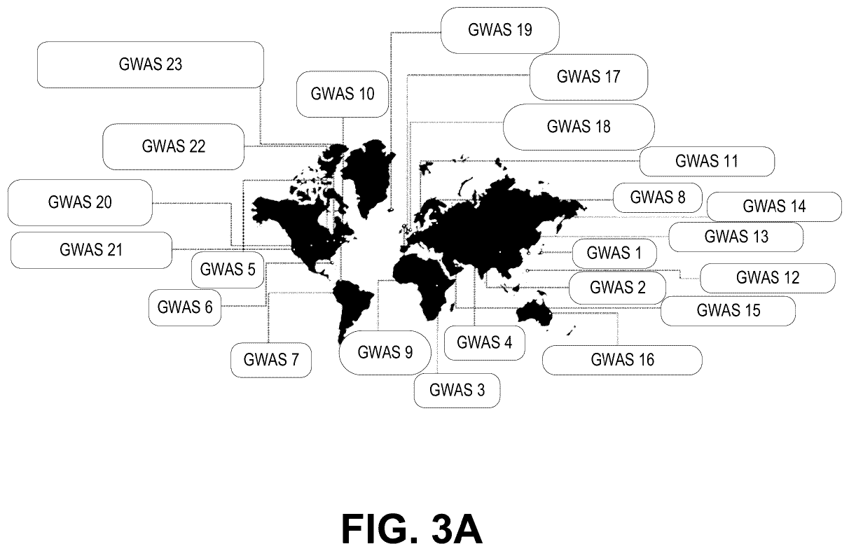

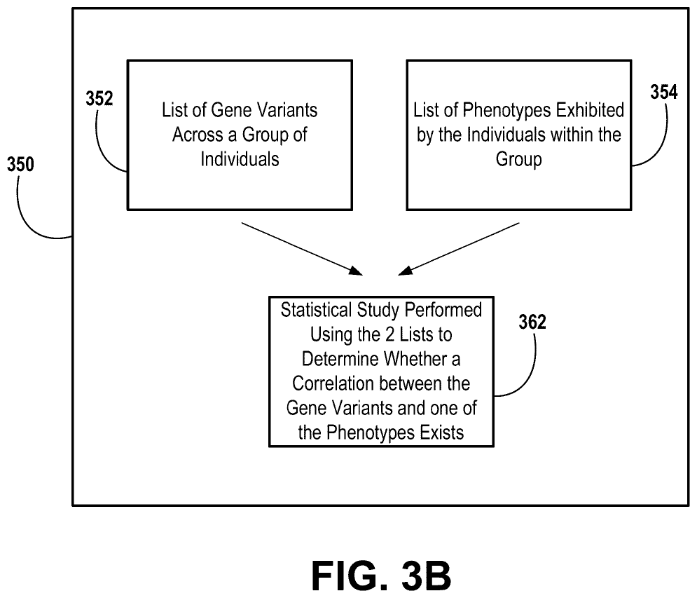

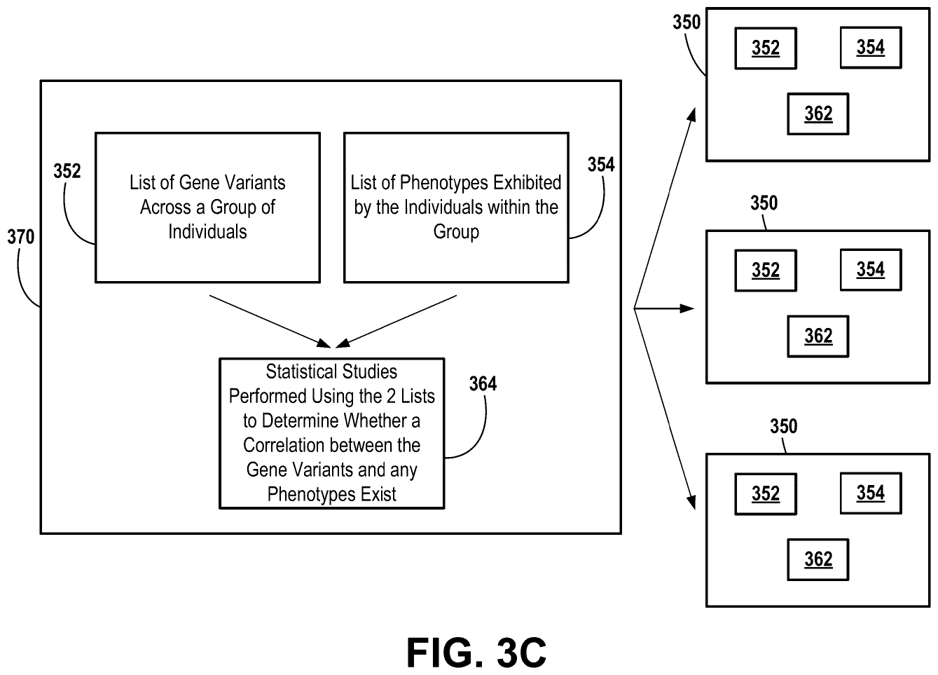

FIG. 3A illustrates a set of genetic studies performed in different locations throughout the world. In particular, FIG. 3A illustrates twenty three different GWASs. Because such genetic studies may be performed on disparate genetic populations (e.g., with different underlying phenotypes) and/or may analyze different genetic variants/different phenotypes, such genetic studies may include different underlying results. As described above, the techniques described herein may allow the results and/or the inherent data from different genetic studies to be combined. For example, the data contained in GWAS 7 and GWAS 16 may be combined to yield additional information about the underlying genetic relationships between one or more genetic variants and a specified genetic disorder. To allow for such combinations to be made, the techniques disclosed herein may include one or more quality assessments and/or one or more merging techniques. For instance, data related to GWAS 7 and GWAS 16 may be received at a computing system. The computing system (e.g., a processor of the computing system) may then perform a quality assessment on both GWAS 7 and GWAS 16. After both GWAS 7 and GWAS 16 pass the quality assessment, data from GWAS 7 and GWAS 16 may be merged using a merging technique (e.g., which may allow for subsequent analysis of the combined data).

FIG. 3B is an illustration of a genetic study 350, according to example embodiments. The genetic study 350 may include a GWAS, for example. Further, in some embodiments, the genetic study 350 may be stored (e.g., within a memory, such as a non-volatile memory) as one or more files and/or in one or more databases. As illustrated, the genetic study 350 may include a list of gene variants 352 from across a group of individuals (e.g., sampled from a specified population). The gene variants may have been measured using one or more genetic screenings or tests (e.g., a complete deoxyribonucleic acid (DNA) sequencing). The list of gene variants 352 may include a variety of pieces of metadata used to describe each gene variant. For example, the list of gene variants 352 may include variant identifiers that indicate names and/or chromosomal locations of the gene variants. In some embodiments, for instance, the variant identifiers may include a chromosome, position, reference allele, and alternative allele (CPRA) identification and/or reference SNP cluster identifiers (rsIDs). The CPRA identification may be based on a current Genome Research Consortium human (GRCh) build (e.g., the GRCh38). The list of gene variants 352 may also include a number of occurrences of the respective gene variant, one or more p-values (e.g., which capture the probabilities of the identified degree of correlation occurring between the given gene variant and a specified phenotype if, in fact, no such association exists), and/or one or more standard errors (e.g., associated with statistical inferences made between the given gene variant and a specified phenotype).

Further, the genetic study 350 may also include a list of phenotypes 354 that are exhibited by the individuals within the sampled group. The phenotypes in the list of phenotypes 354 may be determined based on one or more clinical reports, diagnostic tests, and/or electronic health records associated with the individuals. Additionally or alternatively, the phenotypes within the list of phenotypes 354 may have been self-reported by the individuals within the sampled group. For example, an individual may self-report their height, weight, eye color, hair color, incidence of one or more diseases, personal habits (e.g., related to smoking or consuming alcohol), etc.

In some embodiments, the list of gene variants 352 and the list of phenotypes 354 may be linked to one another or otherwise associated with one another (e.g., using one or more lookup tables) such that gene variants within the list of gene variants 352 can be readily associated with phenotypes within the list of phenotypes 354 (e.g., in order to perform statistical analyses of underlying relationships between gene variants and phenotypes). Similarly, the genetic study 350 may include a statistical study 362 (e.g., analysis) that was previously performed and recorded. The statistical study 362 may be linked to one or more of the gene variants from the list of gene variants 352 and one of the phenotypes from the list of phenotypes 354. For example, the statistical study 362 may include data that represents a series of statistical determinations made regarding whether any of the gene variants within the list of gene variants 352 are associated with a single phenotype within the list of phenotypes 354. In some embodiments, the statistical study 362 may include multiple pieces of metadata that describes the study, such as a study identifier, a phenotype studied (e.g., contained within the list of phenotypes 354), the number of gene variants from the list of gene variants 352 that were considered as part of the statistical study 362, the sample size (e.g., the number of individuals that participated in the genetic study 350 who were considered as part of the statistical study 362), whether whole-exome data was considered as part of the statistical study 362 (e.g., as opposed to whole-genome data), etc.

Understandably, because the statistical study 362 relates one or more gene variants within the list of gene variants 352 to one of the phenotypes in the list of phenotypes 354, it may be desirable to perform a series of statistical studies that analyze multiple phenotypes within the list of phenotypes 354. FIG. 3C illustrates a genetic study 370 that includes multiple statistical studies 364. Like the genetic study 350 illustrated in FIG. 3B, the genetic study 370 includes the list of gene variants 352 and the list of phenotypes 354. However, instead of a single statistical study 362, the genetic study 370 includes a series of statistical studies 364 (e.g., stored within a list or other data structure). In some embodiments, the genetic study 370 may represent a GWAS (e.g., one of the GWASs illustrated in FIG. 3A). In some embodiments, each of the statistical studies 364 may correspond to a different phenotype within the list of phenotypes 354. While the genetic study 370 may be stored (e.g., within a memory, such as a non-volatile memory) as a single entity (e.g., as data relating to a single GWAS), it is understood that such data could be readily separable (e.g., by a computing system, such as the computing system 100 shown and described with reference to FIGS. 1 and 2). For example, as illustrated in FIG. 3C, the genetic study 370 that includes multiple statistical studies 364 (e.g., of multiple phenotypes within the list of phenotypes 354) may be considered as and/or separated into a set of genetic studies 350 that each only include a single statistical study 362 (e.g., of a single phenotype within the list of phenotypes 354), like the genetic study 350 shown and described with reference to FIG. 3B.

It is understood that techniques described herein could be performed on either the genetic study 350 shown and described with reference to FIG. 3B or the genetic study 370 shown and described with reference to FIG. 3C. In some embodiments, for example, a computing system (e.g., the computing system 100 shown and described with reference to FIG. 1) may receive one or more genetic studies 370 and perform the quality assessments and merging techniques disclosed herein using the data from the genetic studies 370. For instance, the genetic study 370 illustrated in FIG. 3C may represent a candidate data set to be analyzed (e.g., and, potentially, merged with another candidate data set).

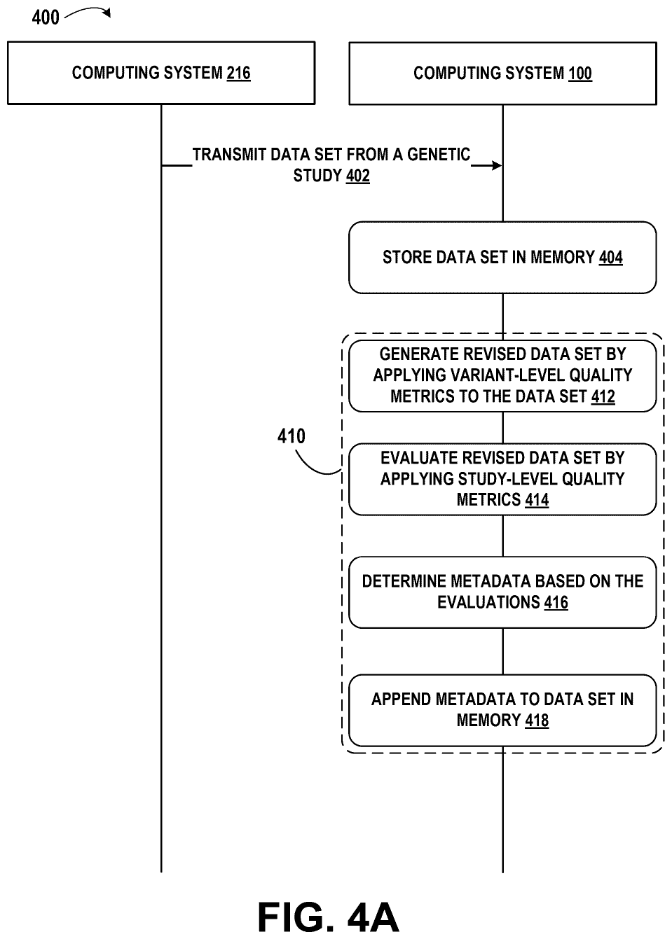

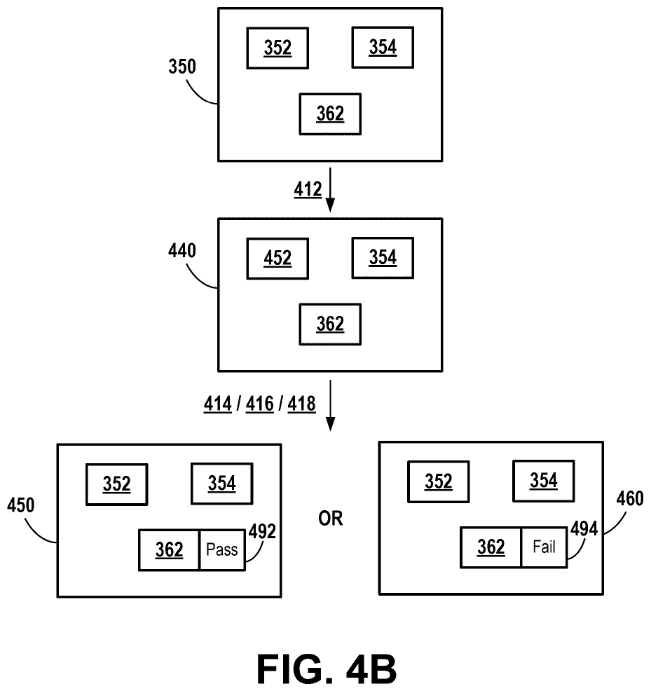

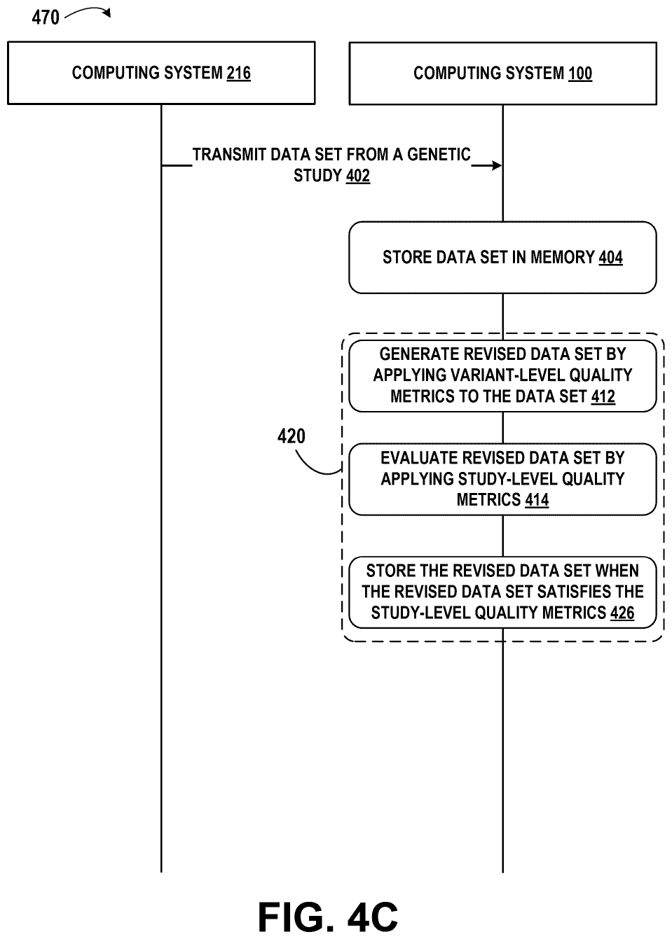

FIG. 4A is a communication flow diagram of a network (e.g., components of the network 200 shown and described with reference to FIG. 2). The network may execute a process 400 (e.g., to perform a quality assessment 410 on a candidate data set). As illustrated, the process 400 may include communications between and actions of two computing systems (e.g., the computing system 100 shown and described with reference to FIG. 1 and FIG. 2 and the computing system 216 shown and described with reference to FIG. 2). It is understood that, in some embodiments, devices or systems other than those illustrated in FIG. 4A may participate in a process similar to the one illustrated in FIG. 4A. For example, rather than the computing system 216, the computing system 100 may communicate with a cloud service (e.g., the cloud service 214 shown and described with reference to FIG. 2) or a server (e.g., the server 212 shown and described with reference to FIG. 2). For instance, in some embodiments, the data set may be stored within a genetics repository associated with a subscription service that is accessed by the computing system 100 (e.g., using login credentials) in order to retrieve the data set for the quality assessment 410. In still other embodiments, no communication between devices on a network may be performed in order to complete the quality assessment 410 (e.g., a processor 102 of the computing system 100 may simply retrieve a candidate data set from a memory of the computing system 100, such as the fixed mass storage device 120, and perform the quality assessment 410 on the retrieved candidate data set).

As illustrated in FIG. 4A, at step 402, the process 400 may include the computing system 216 transmitting a data set from a genetic study (e.g., a GWAS conducted within a population) to the computing system 100 (e.g., to a processor 102 of the computing system 100 via a network interface 116). In some embodiments, the transmission of step 402 may be performed by the computing system 216 in response to a request from the computing system 100 (e.g., a request for a candidate data set). In some embodiments, step 402 may include the computing system 216 transmitting a genetic study (e.g., like the genetic study 370 shown and described with reference to FIG. 3C) that includes a list of gene variants 352, a list of phenotypes 354, and results of one or more statistical studies 364.

At step 404, the process 400 may include the computing system 100 (e.g., a processor 102 of the computing system 100) storing the data set within a memory (e.g., the memory 110, the removable mass storage device 112, and/or the fixed mass storage device 120). For example, the data set may be stored within a volatile memory for rapid access by the processor 102 and/or within a non-volatile memory for long-term storage (e.g., for later analysis).

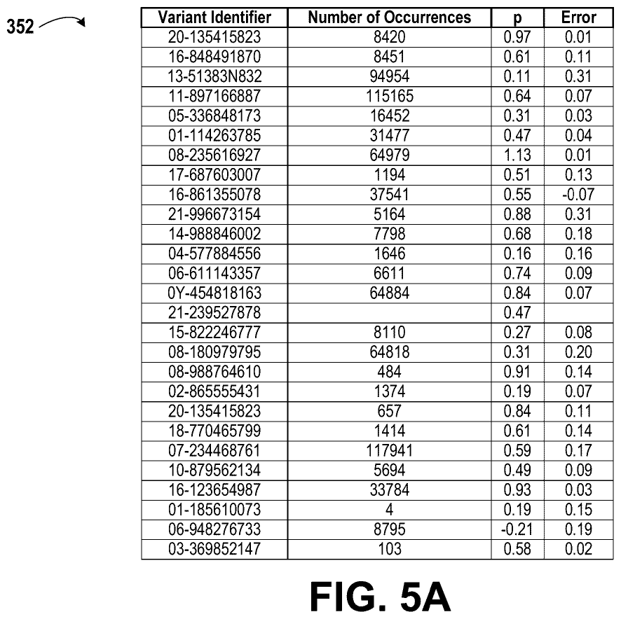

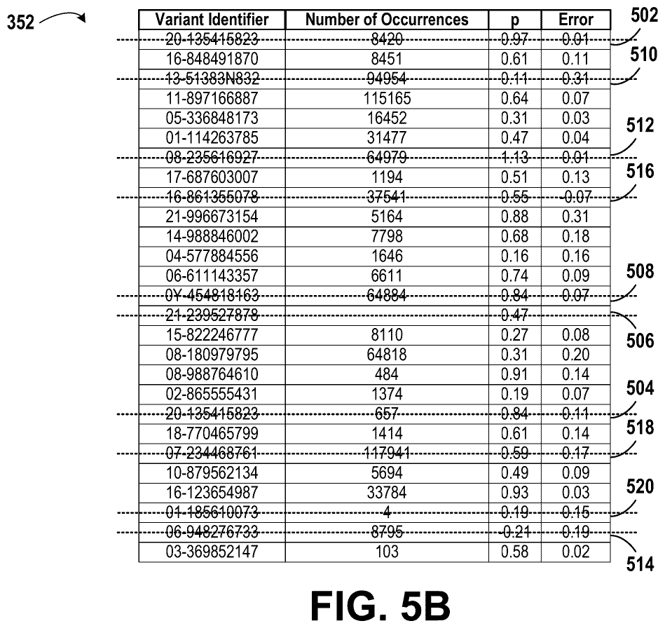

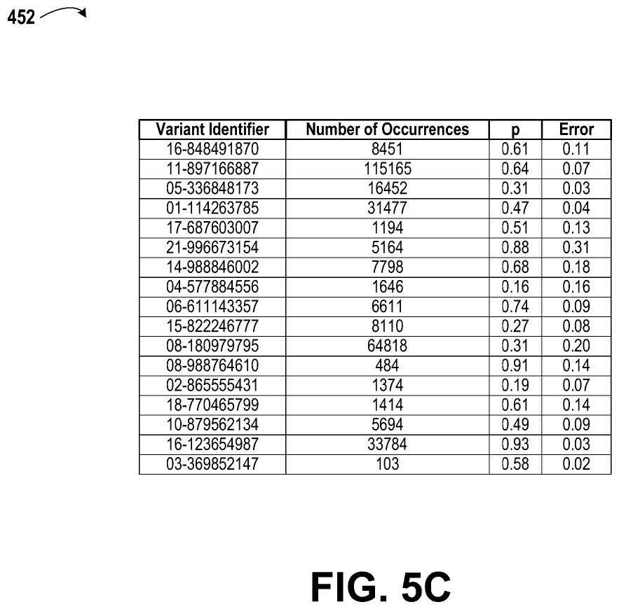

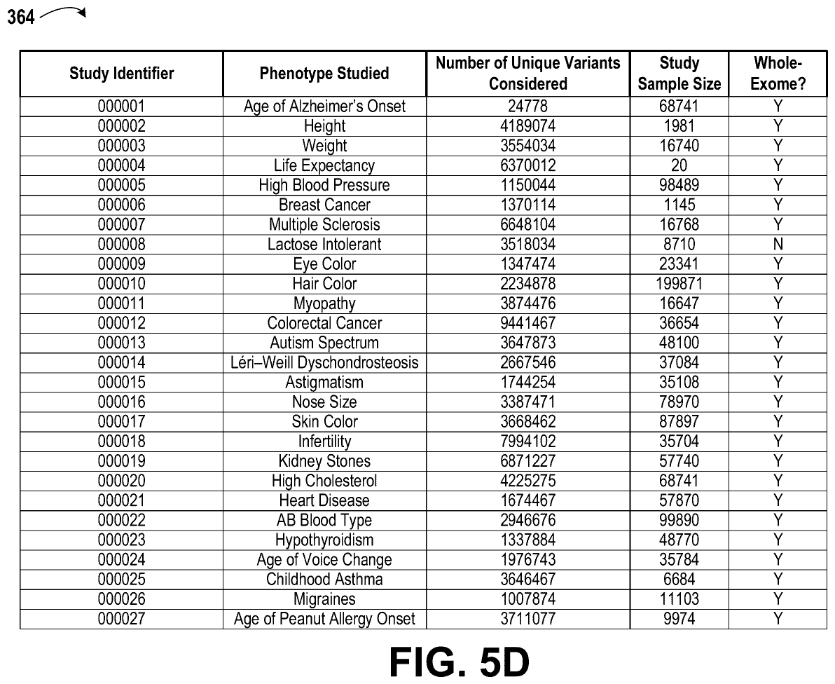

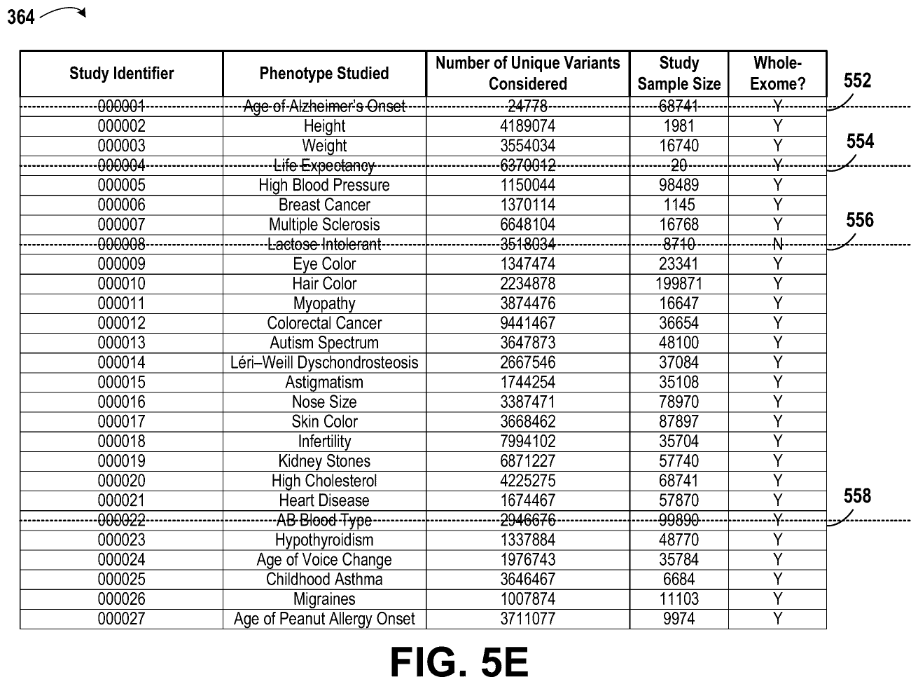

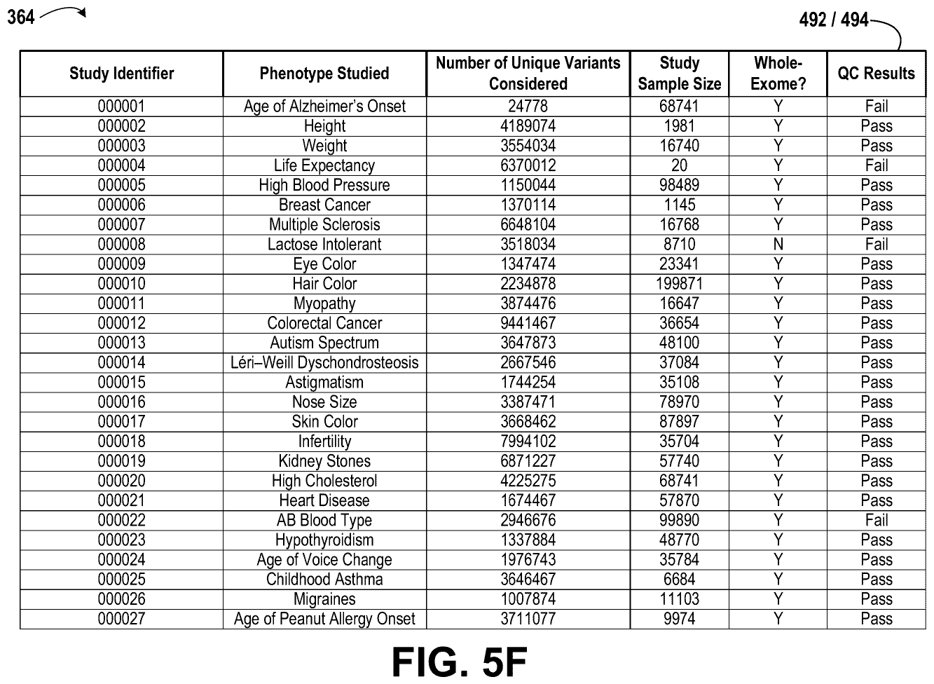

Thereafter, the process 400 may include the computing system 100 (e.g., a processor 102 of the computing system 100) performing a quality assessment 410. The quality assessment 410 may be performed to determine the quality of the data set that was stored in memory at step 404. The quality assessment 410 may, itself, include multiple steps. For example, at step 412 the quality assessment 410 may include the computing system 100 (e.g., a processor 102 of the computing system 100) generating a revised data set by applying variant-level quality metrics to the data set transmitted in step 402 and received in step 404. Applying variant-level quality metrics may include identifying and removing unacceptable gene variants within the data set. Example variant-level quality metrics are shown and described further with reference to FIGS. 5A-5C.

After removing the unacceptable gene variants from the data set to generate the revised data set, at step 414 the quality assessment 410 may include the computing system 100 (e.g., a processor 102 of the computing system 100) evaluating the revised data set by applying one or more study-level quality metrics. Applying study-level quality metrics may include determining whether each of the one or more statistical studies 364 (e.g., either as initially performed or as revised based on the revised set of gene variants after applying the variant-level quality metrics) are of sufficient quality. In some embodiments (e.g., as shown and described with reference to FIG. 4C), evaluating the revised data set by applying study-level quality metrics may include removing those studies that do not satisfy the one or more study-level quality metrics. Example study-level quality metrics are shown and described further with reference to FIG. 5D-5F.

Once the study-level quality metrics have been applied, at step 416 the quality assessment 410 may include the computing system 100 (e.g., a processor 102 of the computing system 100) determining metadata for the data set based on the evaluations of step 414. For example, metadata may be determined for one or more gene variants in the original data set. Each piece of variant metadata may indicate whether or not the respective gene variant satisfied each of the one or more variant-level quality metrics. Additionally or alternatively, metadata may be determined for one or more studies in the original data set. Each piece of study metadata may indicate whether or not the respective study satisfied each of the one or more study-level quality metrics.

At step 418, the quality assessment 410 may include appending metadata (e.g., the metadata determined in step 416) to the data set that was transmitted at step 402 and stored at step 404. Though not illustrated in FIG. 4A, it is understood that, in some embodiments, in addition to or instead of determining metadata at step 416 and appending that metadata at step 418, the revised data set may be stored within a memory (e.g., after removing unacceptable gene variants by applying one or more variant-level quality metrics and after removing unacceptable studies by applying one or more study-level quality metrics). For example, the revised data set may be stored within a memory of the computing system 100 (e.g., the memory 110, the removable mass storage device 112, and/or the fixed mass storage device 120). Additionally or alternatively, the revised data set may be stored within an auxiliary memory (e.g., a memory associated with a server 212 as shown and described with reference to FIG. 2, a memory associated with a cloud service 214 as shown and described with reference to FIG. 2, or a memory associated with the computing system 216). In some embodiments, the revised data set may also be appended with metadata prior to storage within a memory or an auxiliary memory.

FIG. 4B illustrates a quality assessment (e.g., the quality assessment 410 shown and described with reference to FIG. 4A) in terms of modifications to data sets. For example, as illustrated, the quality assessment may start with a data set. For instance, the quality assessment may receive the genetic study 350 shown and described with reference to FIG. 3B, which includes the list of gene variants 352, the list of phenotypes 354, and the statistical study 362. After applying the variant-level quality metrics (e.g., step 412 shown and described above with reference to FIG. 4A), a revised data set 440 may be generated. The revised data set 440 may include the same list of phenotypes 354 and statistical study 362 as the genetic study 350. However, the revised data set 440 may include a revised list of gene variants 452 (as opposed to the list of gene variants 352 of the genetic study 350). Though not illustrated, it is understood that in some embodiments, the statistical study 362 could be reperformed using the revised list of gene variants 452 during step 412, meaning the revised data set 440 would include a revised statistical study (rather than statistical study 362, as illustrated).

The one or more study-level quality metrics may then be applied to the revised data set 440 (e.g., step 414 shown and described above with reference to FIG. 4A) and metadata may be determined and appended to the original genetic study 350 (e.g., steps 416 and 418 shown and described above with reference to FIG. 4A). This may result in a passing data set 450 or a failing data set 460. As illustrated, a passing data set 450 may include metadata indicative of a pass 492 of the study-level quality metrics appended to the statistical study 362, whereas a failing data set 460 may include metadata indicative of a fail 494 of the study-level quality metrics appended to the statistical study 362. While FIG. 4B illustrates a quality assessment performed from the genetic study 350 shown and described with reference to FIG. 3B, it is understood that a quality assessment could be performed starting from the genetic study 370 shown and described with reference to FIG. 3C (i.e., could be performed on a data set that includes multiple statistical studies). In embodiments with multiple statistical studies, after generating the revised list of gene variants 452, a separate piece of pass/fail metadata may be determined for and appended to each of the statistical studies of the genetic study 370.

FIG. 4C is a communication flow diagram of a network (e.g., components of the network 200 shown and described with reference to FIG. 2). The network may execute a process 470 (e.g., to perform a quality assessment 420 on a candidate data set). As illustrated, the process 470 may include communications between and actions of two computing systems (e.g., the computing system 100 shown and described with reference to FIG. 1 and FIG. 2 and the computing system 216 shown and described with reference to FIG. 2). It is understood that, in some embodiments, devices or systems other than those illustrated in FIG. 4C may participate in a process similar to the one illustrated in FIG. 4C. For example, rather than the computing system 216, the computing system 100 may communicate with a cloud service (e.g., the cloud service 214 shown and described with reference to FIG. 2) or a server (e.g., the server 212 shown and described with reference to FIG. 2). For instance, in some embodiments, the data set may be stored within a genetics repository associated with a subscription service that is accessed by the computing system 100 (e.g., using login credentials) in order to retrieve the data set for the quality assessment 420. In still other embodiments, no communication between devices on a network may be performed in order to complete the quality assessment 420 (e.g., a processor 102 of the computing system 100 may simply retrieve a candidate data set from a memory of the computing system 100, such as the fixed mass storage device 120, and perform the quality assessment 420 on the retrieved candidate data set).

Like the process 400 shown and described with reference to FIG. 4A, the process 470 of FIG. 4C may include computing system 216 transmitting a data set from a genetic study (e.g., a GWAS conducted within a population) to the computing system 100 (e.g., to a processor 102 of the computing system 100 via a network interface 116). In some embodiments, the transmission of step 402 may be performed by the computing system 216 in response to a request from the computing system 100 (e.g., a request for a candidate data set). In some embodiments, step 402 may include the computing system 216 transmitting a genetic study (e.g., like the genetic study 370 shown and described with reference to FIG. 3C) that includes a list of gene variants 352, a list of phenotypes 354, and results of one or more statistical studies 364.

Also like the process 400 shown and described with reference to FIG. 4A, at step 404, the process 400 may include the computing system 100 (e.g., a processor 102 of the computing system 100) storing the data set within a memory (e.g., the memory 110, the removable mass storage device 112, and/or the fixed mass storage device 120). For example, the data set may be stored within a volatile memory for rapid access by the processor 102 and/or within a non-volatile memory for long-term storage (e.g., for later analysis).

Thereafter, the process 470 may include the computing system 100 (e.g., a processor 102 of the computing system 100) performing a quality assessment 420. Like the quality assessment 410 shown and described with reference to FIG. 4A, the quality assessment 420 may be performed to determine the quality of the data set that was stored in memory at step 404. However, the quality assessment 420 of the process 470 of FIG. 4C may include slightly different steps than the quality assessment 410 of the process 400 of FIG. 4A.

Like the quality assessment 410 of the process 400 of FIG. 4A, at step 412 the process 470 may include the computing system 100 (e.g., a processor 102 of the computing system 100) generating a revised data set by applying variant-level quality metrics to the data set transmitted in step 402 and received in step 404. Applying variant-level quality metrics may include identifying and removing unacceptable gene variants within the data set. Example variant-level quality metrics are shown and described further with reference to FIGS. 5A-5C.

Also like the quality assessment 410 of the process 400 of FIG. 4A, after removing the unacceptable gene variants from the data set to generate the revised data set, at step 414 the process 470 may include the computing system 100 (e.g., a processor 102 of the computing system 100) evaluating the revised data set by applying one or more study-level quality metrics. Further, evaluating the revised data set at step 414 of the quality assessment 420 of FIG. 4C may include removing, altogether, those studies (e.g., those statistical studies 364) that do not satisfy the one or more study-level quality metrics. After removing the studies that do not satisfy the one or more study-level quality metrics, the revised data set may include only gene variants that satisfy the one or more variant-level quality metrics and only studies that satisfy the one or more study-level quality metrics.

Thereafter, at step 426 the quality assessment 420 may include storing the revised data set when at least one of the one or more studies satisfies the study-level quality metrics (i.e., when the revised data set still includes at least one statistical study after the studies that do not satisfy the one or more study-level quality metrics have been removed). The revised data set may be stored within a memory of the computing system 100 (e.g., the memory 110, the removable mass storage device 112, and/or the fixed mass storage device 120) or an auxiliary memory (e.g., a memory of the computing system 216, a memory associated with a server 212, a memory associated with a cloud service 214, etc.).

Because the process 470 shown and described with reference to FIG. 4C may include storing the revised data set (rather than appending metadata as shown and described with respect to the process 400 of FIG. 4A), the process 470 may make performing subsequent analyses (e.g., at a later date) using the revised data set quicker (e.g., because the one or more variant-level and/or study-level quality metrics may not be reapplied to eliminate those unacceptable gene variants/studies). However, along the same lines, the process 470 may result in the consumption of additional computing resources (e.g., memory space) for extended storage (e.g., when compared to the process 400 of FIG. 4A since the data set and the revised data set may both be stored after the process 470 of FIG. 4C).

FIG. 5A is an illustration of a list of gene variants (e.g., the list of gene variants 352 shown and described with reference to FIGS. 3B and 3C) in a candidate data set (e.g., the data set transmitted by the computing system 216 and received by the computing system 100 at step 402 of the process 400 shown and described with reference to FIG. 4A). For example, the list of gene variants 352 shown in FIG. 5A may be the state of the list prior to an application of one or more variant-level quality metrics to remove unacceptable gene variants from the list of gene variants 352. As illustrated, each of the gene variants in the list of gene variants 352 may include multiple pieces of variant-level metadata, such as a variant identifier, a number of occurrences, a p-value, and an error value. It is understood that the variant-level metadata provided in FIG. 5A for each gene variant is an example and that additional or alternative variant-level metadata may also be provided for each gene variant within a list of gene variants 352. Further, in some embodiments, one or more pieces of the variant-level metadata provided in FIG. 5A may not be provided within the list of gene variants 352.

The variant identifier may be used to describe the specified gene variant (e.g., by name; by locus; by type of variation; according to a variant taxonomy, such as GRCh38; etc.). The number of occurrences may be used to describe the number of times the specified gene variant occurred within the underlying data set (e.g., within the individuals studied to produce the data set). The p-value may represent the probability of getting an associated statistical result (e.g., within an associated statistical study 362 of a given phenotype from the list of phenotypes 354) if there were in fact no underlying association between the gene variant and the studied phenotype. In some embodiments, there may be multiple p-values listed for one or more of the gene variants (e.g., if a gene variant's potential associations with multiple phenotypes were studied within the data set). The error may represent the statistical error of a statistical determination made for the specified gene variant (e.g., for an associated statistical study 362 of a given phenotype from the list of phenotypes 354). For example, the error may correspond to the size of a confidence interval associated with a statistical determination. In some embodiments, there may be multiple error values listed for one or more of the gene variants (e.g., if a gene variant's potential associations with multiple phenotypes were studied within the data set).

FIG. 5B illustrates the application of variant-level quality metrics to the list of gene variants 352, according to example embodiments. For example, FIG. 5B may correspond to step 412 shown and described with reference to FIGS. 4A and 4C. After applying the variant-level quality metrics, one or more gene variants within the list may be selected for removal from the list (indicated by the strikethroughs of FIG. 5B). For example, gene variants 502 and 504 may have been identified for removal because they have the same variant identifier. Even though the variant-level metadata is not identical for gene variants 502 and 504, these gene variants may nonetheless be tagged for removal. Additionally or alternatively, in some embodiments, any gene variants that have identical variant-level metadata may also be tagged for removal. Though not illustrated, it is also understood that, in alternative embodiments, rather than removing both gene variant 502 and gene variant 504, only one of the two may be tagged for removal. In still other embodiments, gene variant 502 and gene variant 504 may be merged rather than either being tagged for removal. Further, gene variant 506 may be tagged for removal because it has an incomplete set of variant-level metadata (e.g., gene variant 506 is missing the number of occurrences and the error variant-level metadata). In addition, gene variant 508 may be tagged for removal because it is associated with a chromosome other than chromosomes 1-22 and the X chromosome (e.g., as indicated by the ‘Y’ in the variant identifier, gene variant 508 may be associated with the Y chromosome and, therefore, has been tagged for removal). Still further, gene variant 510 may be tagged for removal because it is associated with a gene variant for which the variant identifier is missing one or more pieces of information defined by the variant taxonomy (e.g., as indicated by the ‘N’ in the variant identifier). Even further, gene variants 512 and 514 may be tagged for removal because they have invalid p-values (e.g., because gene variants 512 and 514 have p-values that are not between 0 and 1, inclusive). Yet further, gene variant 516 may be tagged for removal because it has an invalid error value (e.g., because gene variant 516 has an error value that is not greater than or equal to 0). Yet even further, gene variant 518 may be tagged for removal because it corresponds to a qualitative (as opposed to a quantitative) trait. This may be determined based on the locus of gene variant 518 (e.g., as indicated by the variant identifier) and/or based on another piece of variant-level metadata (e.g., an indication of a phenotype that corresponds to gene variant 518, which is not illustrated in FIG. 5B).

Even still further, gene variant 520 may be tagged for removal because it doesn't have a minimum threshold number of occurrences (e.g., 5 or more). In various embodiments, the minimum threshold number of occurrences may be determined using various techniques. For example, the gene variant 520 may be matched to a maximally analogous gene variant within another data set (e.g., another list of gene variants within another data set being analyzed by the computing system 100). Then, the minor allele frequency associated with the maximally analogous gene variant may be used to determine the minimum threshold number of occurrences (e.g., the minimum threshold number of occurrences may be two times the minor allele frequency times a number of individuals within the population studied within the underlying genetic study 350).

After identifying those gene variants that should be removed from the list of gene variants 352, the computing system 100 may remove the unacceptable gene variants (e.g., those gene variants highlighted above with reference to FIGS. 4A and 4B). This may result in the revised list of gene variants 452 (e.g., as shown and described with reference to FIG. 4B). FIG. 5C illustrates an example revised list of gene variants 452, according to example embodiments.

FIG. 5D is an illustration of a list of statistical studies (e.g., the statistical studies 364 shown and described with reference to FIG. 3C) in a candidate data set (e.g., the data set transmitted by the computing system 216 and received by the computing system 100 at step 402 of the process 400 shown and described with reference to FIG. 4A). For example, the list of statistical studies 364 shown in FIG. 5D may be the state of the list prior to an application of one or more study-level quality metrics to append metadata indicating a failure to unacceptable studies from the list of statistical studies 364. As illustrated, each of the studies in the list of statistical studies 364 may include multiple pieces of study-level metadata, such as a study identifier, a phenotype studied, a number of unique variants considered for the study, a sample size of the study, and whether the study considered whole-exome data. It is understood that the study-level metadata provided in FIG. 5D for each statistical study 364 is an example and that additional or alternative study-level metadata may also be provided for each statistical study within a list of statistical studies 364. Further, in some embodiments, one or more pieces of the study-level metadata provided in FIG. 5D may not be provided within the list of statistical studies 364.

The study identifier may be used to identify the specific statistical study (e.g., by name; by type of statistical study; by type of analysis performed; according to a study taxonomy; etc.). The phenotype studied may be used to identify which phenotype (e.g., from the list of phenotypes 354) was considered for potential gene variant association. The number of unique variants considered may be used to identify how many gene variants (e.g., what number of gene variants within the list of gene variants 352) were considered in the study. The study sample size may be used to identify how many individuals (e.g., how many human subjects) were considered in the study (e.g., based on the number of individuals for which data was collected for an associated underlying data set). Whether the study considered whole-exome data may be used to identify whether whole-exome data or some other type of gene variant data (e.g., whole-genome data) was considered for the study.

FIG. 5E illustrates the application of study-level quality metrics to the list of statistical studies 364, according to example embodiments. For example, FIG. 5E may correspond to step 414 shown and described with reference to FIGS. 4A and 4C. After applying the study-level quality metrics, one or more statistical studies 364 within the list may be selected for appending pass/fail metadata (selected for appending fail metadata indicated by the strikethroughs of FIG. 5E). This may be similar to the appending of metadata shown and described in FIG. 4B, for example. Additionally or alternatively, after applying the study-level quality metrics, one or more statistical studies 364 within the list may be selected for removal from the list and from the revised data set.

As illustrated in FIG. 5E, study 552 may have been identified for appending metadata indicating a failed study-level quality metric. Study 552 may have been tagged for appending metadata indicating a failed study-level quality metric because the number of unique variants considered does not meet a minimum threshold number of unique gene variants within the genetic study (e.g., 1 million or more). Study 554 may have been tagged for appending metadata indicating a failed study-level quality metric because the study sample size does not meet a minimum sample size (e.g., a minimum threshold sample size, such as 100, 500, 1,000, 10,000, 50,000, etc.) within the genetic study (e.g., the associated genetic study 370). Study 556 may have been tagged for appending metadata indicating a failed study-level quality metric because the study does not consider whole-exome data. Study 558 may have been tagged for appending metadata indicating a failed study-level quality metric because the phenotype studied corresponds to a qualitative trait (as opposed to a quantitative trait). Oppositely, in some embodiments, instead of tagging qualitative traits, quantitative traits may be tagged. In other embodiments, some hybrid consideration of various study-level metadata may be performed. For example, if a qualitative trait (e.g., a case-control trait) is being considered in a study, the study sample size required to avoid being tagged may be higher (e.g., 2,000 individuals) than for a quantitative trait (e.g., 1,000 individuals). Alternatively, if a qualitative trait (e.g., a case-control trait) is being considered in a study, the study sample size may be defined differently (e.g., the number of “case” individuals exhibiting the phenotype and the number of “control” individuals not exhibiting the phenotype) than for a quantitative trait (e.g., the total number of individuals considered). Hence, the thresholds to avoid tagging may be defined in different manners depending on the type of study (e.g., a case-control study may include a threshold number of case individuals and a threshold number of control individuals, whereas a quantitative study may include only a threshold number of total individuals).

Though not illustrated in FIG. 5E, additional or alternative study-level metrics may be applied to the studies to identify one or more studies to be tagged for appending metadata indicating a failed study-level quality metric. For example, for case-control studies, a logarithm of an odds ratio (e.g., for a given phenotype) for each gene variant in the reduced set of gene variants (e.g., reduced based on the application of the variant-level quality metrics) may be calculated. Thereafter, a first quartile odds ratio and a third quartile odds ratio may be determined. The first quartile odds ratio may be compared with a first threshold value (e.g., −0.2 or less) and the third quartile odds ratio may be compared with a second threshold value (e.g., 0.2 or more). If the first quartile odds ratio is less than the first threshold value or the third quartile odds ratio is greater than the second threshold value, the study may be tagged for appending metadata indicating a failed study-level quality metric.

In some embodiments, in order to identify one or more studies to be tagged for appending metadata indicating a failed study-level quality metric, a chi-squared test (e.g., for a given phenotype) for each of the gene variants in the reduced set of gene variants (e.g., reduced based on the application of the variant-level quality metrics) may be calculated to determine a set of chi-squared values. A median chi-squared value from among the chi-squared values may then be determined. A genomic inflation factor may then be determined by dividing the median chi-squared value by an expected median of a chi-squared distribution with an appropriate corresponding number of degrees of freedom. This genomic inflation factor may be compared to a minimum threshold genomic inflation factor (e.g., 0.9 or more) and a maximum threshold genomic inflation factor (e.g., 1.5 or less). If the genomic inflation factor is not within the range spanned by the minimum threshold genomic inflation factor and the maximum threshold genomic inflation factor (i.e., if the genomic inflation factor is less than the minimum threshold genomic inflation factor or greater than the maximum threshold genomic inflation factor), the study may be tagged for appending metadata indicating a failed study-level quality metric.

Additionally or alternatively, the determined genomic inflation factor may be normalized to the genomic inflation factor of a study (e.g., a case-control study) having 1000 cases and 1000 controls (or some other number of cases/controls) to determine a normalized genomic inflation factor. Thereafter, the normalized genomic inflation factor may be compared to a maximum threshold normalized genomic inflation factor (e.g., 1.5 or less). If the normalized genomic inflation factor is greater than the maximum threshold normalized genomic inflation factor, the study may be tagged for appending metadata indicating a failed study-level quality metric.

In some embodiments, in order to identify one or more studies to be tagged for appending metadata indicating a failed study-level quality metric, a minor allele frequency may be determined for each of the gene variants in the reduced set of gene variants (e.g., reduced based on the application of the variant-level quality metrics) based on number of occurrences. The gene variants may then be separated by minor allele frequencies into a plurality of frequency bins (e.g., two frequency bins, three frequency bins, four frequency bins, five frequency bins, six frequency bins, seven frequency bins, eight frequency bins, nine frequency bins, ten frequency bins, eleven frequency bins, twelve frequency bins, thirteen frequency bins, fourteen frequency bins, fifteen frequency bins, etc.). Then, for each frequency bin independently, a chi-squared test for each of the gene variants in the respective frequency bin relative to the other gene variants in the frequency bin may be performed. The median chi-squared value for each of the frequency bins may also be determined and, based on these median chi-squared values, normalized genomic inflation factors may also be determined for each of the frequency bins (e.g., by determining a genomic inflation factor by dividing the median values by expected median values based on degrees of freedom and, thereafter, normalizing the genomic inflation factor to a study having a predetermined number of cases and a predetermined number of controls). The normalized genomic inflation factor having the highest value from among all frequency bins may then be divided by the normalized genomic inflation factor having the lowest value from among all frequency bins to determine a normalized genomic inflation factor ratio. This normalized genomic inflation factor ratio may then be compared to a maximum threshold normalized genomic inflation factor ratio (e.g., 2.0 or less). If the normalized genomic inflation factor ratio is greater than the maximum threshold normalized genomic inflation factor ratio, the study may be tagged for appending metadata indicating a failed study-level quality metric.

In some embodiments, in addition to or instead of tagging one or more studies for appending metadata indicating a failed study-level quality metric and after removing the unacceptable gene variants based on one or more variant-level quality metrics, one or more statistical studies may be reperformed (e.g., one or more statistics may be recalculated based on the reduced set of gene variants, as compared to the original calculation(s) that were performed based on all of the gene variants included in the list of gene variants 352). Thereafter, in some embodiments, the study-level quality metrics may be applied to a revised list of studies (e.g., that correspond to the recalculated statistics based on the revised set of gene variants).

After identifying those studies to which metadata indicating a failed study-level quality metric should be appended, the computing system 100 may append metadata indicating a failed study-level quality metric to those studies and may append metadata indicating a passing of all study-level quality metrics to the remaining studies. This may result in a list of statistical studies 364 with appended pass/fail metadata. FIG. 5F illustrates an example list of gene statistical studies 364 with appended pass/fail metadata, according to example embodiments. The column labeled “QC Results” may correspond to either metadata indicative of a pass 492 or metadata indicative of a fail 494 (similar to the metadata shown and described with reference to FIG. 4B), depending on the study. It is understood that FIG. 5F is provided solely as an example and that other embodiments are also possible and are contemplated herein. For example, instead of simply capturing a “pass” or a “fail” of the study-level quality metrics as a whole, the appended metadata may also explicitly indicate which of the study-level quality metrics were not satisfied by a given study. Additionally or alternatively, for study-level quality metrics that include calculating one or more figures of merit and comparing those figures of merit to one or more threshold values, the determined figures of merit may also be appended as metadata.

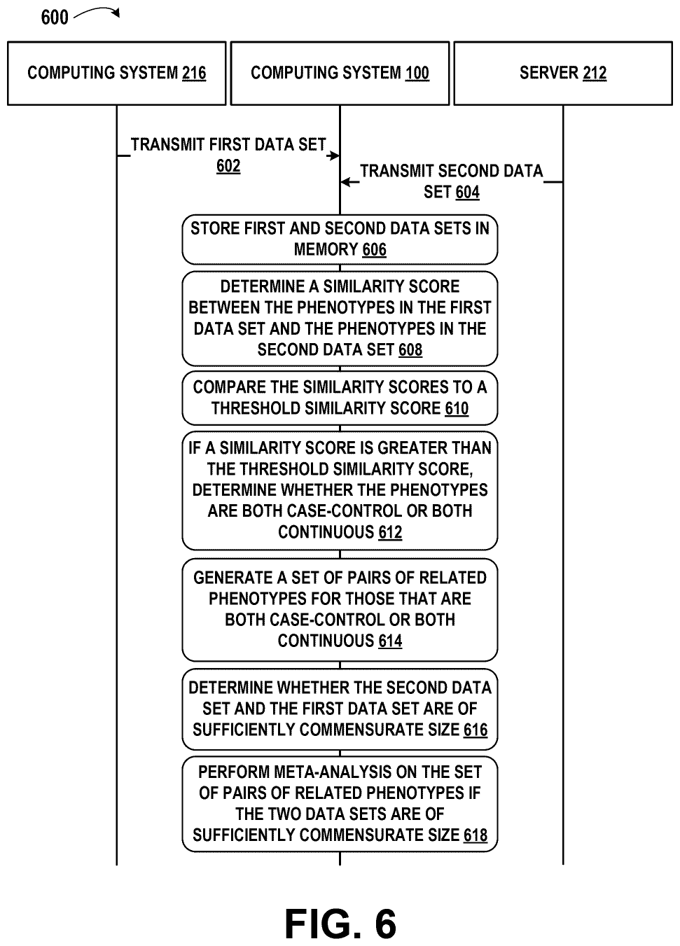

FIG. 6 is a communication flow diagram of a network (e.g., components of the network 200 shown and described with reference to FIG. 2). The network may execute a process 600 (e.g., to perform a merging technique on two data sets). As illustrated, the process 600 may include communications among and actions of two computing systems and a server (e.g., the computing system 100 shown and described with reference to FIG. 1 and FIG. 2, the computing system 216 shown and described with reference to FIG. 2, and the server 212 shown and described with reference to FIG. 2). It is understood that, in some embodiments, devices or systems other than those illustrated in FIG. 6 may participate in a process similar to the one illustrated in FIG. 6. For example, rather than the computing system 216, the computing system 100 may communicate with a cloud service (e.g., the cloud service 214 shown and described with reference to FIG. 2). For instance, in some embodiments, the first data set may be stored within a genetics repository associated with a subscription service that is accessed by the computing system 100 (e.g., using login credentials) in order to retrieve the first data set for the merging technique. Additionally or alternatively, rather than communicating with two other entities, the computing system 100 may instead only communicate with one other entity (e.g., may receive both the first data set and the second data set from the computing system 216 or may receive one of the first data set and the second data set from the computing system 216 and may have the other of the first data set and the second data set already stored within a memory of the computing system 100) and/or may not communicate with any other entities (e.g., may have the first data set and the second data set stored internal to the computing system 100 so that no transmissions are needed).

As illustrated in FIG. 6, at step 602, the process 600 may include the computing system 216 transmitting a first data set from a genetic study (e.g., a GWAS conducted within a population) to the computing system 100 (e.g., to a processor 102 of the computing system 100 via a network interface 116). In some embodiments, the transmission of step 602 may be performed by the computing system 216 in response to a request from the computing system 100 (e.g., a request for the first data set). In some embodiments, the first data set may include a genetic study (e.g., like the genetic study 370 shown and described with reference to FIG. 3C) that includes a list of gene variants 352, a list of phenotypes 354, and results of one or more statistical studies 364.

At step 604, the process 600 may include the server 212 transmitting a second data set from a genetic study (e.g., a GWAS conducted within a population) to the computing system 100 (e.g., to a processor 102 of the computing system 100 via a network interface 116). In some embodiments, the transmission of step 604 may be performed by the server 212 in response to a request from the computing system 100 (e.g., a request for the second data set). In some embodiments, the second data set may include a genetic study (e.g., like the genetic study 370 shown and described with reference to FIG. 3C) that includes a list of gene variants 352, a list of phenotypes 354, and results of one or more statistical studies 364. In some embodiments, the first data set and the second data second may both be received from the same source (e.g., the server 212 or the computing system 216).

At step 606, the process 600 may include the computing system 100 storing the first data set and the second data set in memory (e.g., within the memory 110, the removable mass storage device 112, and/or the fixed mass storage device 120).

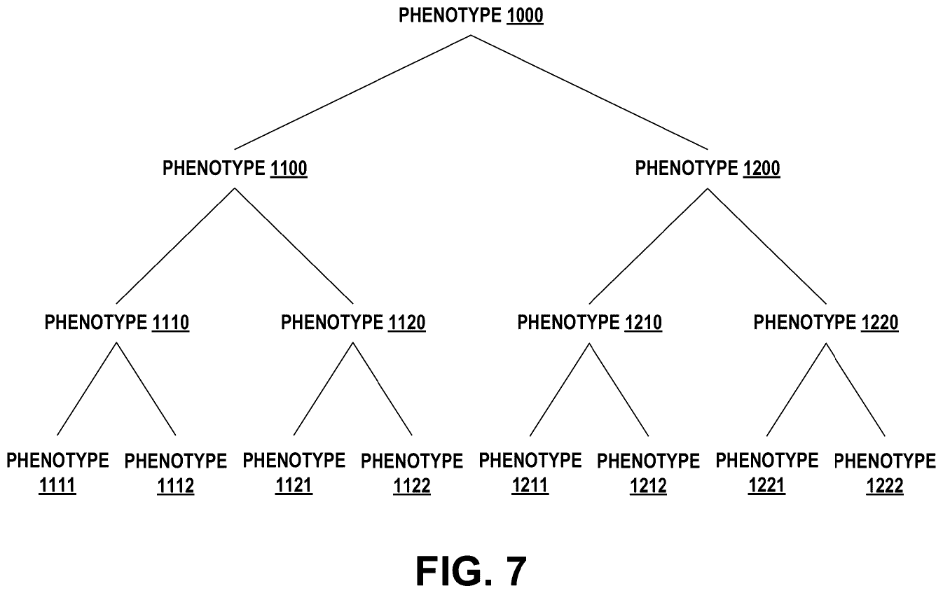

At step 608, the process 600 may include determining, by the computing system 100 (e.g., by the processor 102 of the computing system 100), a similarity score between each of the phenotypes in the first data set and each of the phenotypes in the second data set. Determining a similarity score may include assigning labels to one or more (e.g., each) of the phenotypes in the first data set and/or one or more (e.g., each) of the phenotypes in the second data set. The phenotypes in the second data set may be labeled using the naming convention that was used to establish the labels of the phenotypes in the first data set. Alternatively, the phenotypes in the first data set may be labeled using the naming convention that was used to establish the labels of the phenotypes in the second data set. In some embodiments, assigning labels to one or more of the phenotypes in the first data set or the second data set may include applying a naming ontology to the phenotypes. Further, in some embodiments, in order to assign labels to one or more of the phenotypes, one or more different naming techniques may be applied (e.g., sequentially for each phenotype from a most frequently successful naming technique to a least frequently successful naming technique) until a label is determined for the phenotype. After assigning labels to each of the phenotypes (if necessary), a similarity score may be calculated. Example techniques for calculating a similarity score will be described further below with reference to FIG. 7.

At step 610, the process 600 may include comparing, by the computing system 100 (e.g., by the processor 102 of the computing system 100) each of the similarity scores to a threshold similarity score. The threshold similarity score may be determined based on a desired correspondence between the two data sets (e.g., the stricter the desired correspondence, the higher the threshold similarity score and the more lenient the desired correspondence, the lower the threshold similarity score). Additionally or alternatively, in some embodiments, the similarity score may be determined based on or defined in terms of an ontology used at step 608 to assign one or more of the labels to the phenotypes in the first data set or the second data set. In some embodiments, for example, the threshold similarity score may be 0.95 (e.g., in embodiments using the EFO).

At step 612, the process 600 may include, if a similarity score for a pair of phenotypes is greater than the threshold similarity score, determining, by the computing system 100 (e.g., by the processor 102 of the computing system 100), whether both the phenotype from the first data set and the phenotype from the second data set in the pair of phenotypes are case-control phenotypes (e.g., from a case-control study), both the phenotype from the first data set and the phenotype from the second data set in the pair of phenotypes are continuous phenotypes (e.g., from a quantitative study), or if one phenotype is from a case-control study whereas the other phenotype is from a quantitative study.

At step 614, the process 600 may include generating, by the computing system 100 (e.g., by the processor 102 of the computing system 100), a set of pairs of related phenotypes for those that are both case-control or both continuous. For example, step 614 may include identifying those pairs of phenotypes from the first data set and the second data that both: (i) had a similarity score that exceed the threshold similarity score at step 610 and (ii) were both of the same type (e.g., case-control vs. continuous) at step 612. Those identified pairs may then be stored in a list or other data structure.

At step 616, the process 600 may include determining, by the computing system 100 (e.g., by the processor 102 of the computing system 100), whether the second data set and the first data set are of sufficiently commensurate size (e.g., such that their combination provides non-negligible statistical improvement to one or more studies). This may include determining a ratio between the number of phenotypes considered in the first data set and the second data set, a ratio between the number of individuals studied in the first data set and the second data set, and/or a ratio between the number of studies performed in the first data set and the second data set. For example, step 616 may include calculating a ratio of the number of individuals studied in the smaller data set (e.g., the second data set) to the number of individuals studied in the larger data set (e.g., the first data set). Further, determining whether the second data set and the first data set are of sufficiently commensurate size (e.g., ensuring one data set is not one million times larger than the other), may include comparing the determined ratio to a threshold ratio. The threshold ratio may be 0.1 or more, 0.01 or more, 0.001 or more, 0.0001 or more, 0.00001 or more, etc.

At step 618, the process 600 may include performing, by the computing system 100 (e.g., by the processor 102 of the computing system 100), meta-analysis on the set of pairs of related phenotypes if the two data sets are of sufficiently commensurate size (e.g., if the determined ratio is greater than or equal to threshold ratio). Meta-analysis may include performing additional statistical studies on the combined data, for example.

FIG. 7 is an illustration of a plurality of phenotypes arranged according to an ontology (e.g., the MeSH ontology or EFO), according to example embodiments. As illustrated, the phenotypes may be arranged into a tree structure (e.g., according to the ontology). However, it is understood that the tree structure is provided solely as an example and that other ontological structures are also possible and are contemplated here. As illustrated, phenotype 1000 may be at a root of the tree; phenotype 1100 and phenotype 1200 may be children of phenotype 1000; phenotype 1110 and phenotype 1120 may be children of phenotype 1100 and grandchildren of phenotype 1000; phenotype 1210 and phenotype 1220 may be children of phenotype 1200 and grandchildren of phenotype 1000; phenotype 1111 and phenotype 1112 may be children of phenotype 1110, grandchildren of phenotype 1100, and great-grandchildren of phenotype 1000; phenotype 1121 and phenotype 1122 may be children of phenotype 1120, grandchildren of phenotype 1100, and great-grandchildren of phenotype 1000; phenotype 1211 and phenotype 1212 may be children of phenotype 1210, grandchildren of phenotype 1200, and great-grandchildren of phenotype 1000; and phenotype 1221 and phenotype 1222 may be children of phenotype 1220, grandchildren of phenotype 1200, and great-grandchildren of phenotype 1000. In some embodiments, a parent node may represent a more generalized phenotype (e.g., a genus phenotype) whereas a child node may represent a more specific phenotype (e.g., a species phenotype). For example, phenotype 1000 may represent “cancer,” phenotype 1100 may represent “gastrointestinal system cancer,” phenotype 1110 may represent “colorectal cancer,” and phenotype 1111 may represent “colorectal adenocarcinoma.” Other examples are also possible and are contemplated herein.

Phenotypes arranged according to an ontology (e.g., arranged according to FIG. 7) may be compared with one another to determine a similarity score (e.g., a semantic similarity). For example, such a determination may be made for step 608 of the process 600 described above with reference to FIG. 6. Determining a similarity score between two phenotypes may include performing one or more calculations based on the relative locations of the two phenotypes within the ontology. Various types of calculations may be performed, but two examples are described herein: Lin similarity and Resnik similarity. It is understood that Lin similarity and Resnik similarity are provided solely as examples and that other types of similarity scores and similarity calculations (e.g., calculations tailored to a specific ontology) are also possible and are contemplated herein.

Lin similarity may include determining the information content within each of the two phenotypes, as well as the maximum information content of any common ancestor shared between the two phenotypes. The information content may be defined as:

$$IC ( c ) = - ln ( \frac{a ( c )}{N} )$$

where IC(c) represents the information content of a phenotype c, a(c) represents the number of terms for which c is an ancestor (including itself), and N represents the total number of terms in the ontology. So, considering phenotype 1100 and phenotype 1110 in FIG. 7 as examples, phenotype 1100 has

$$IC ( {c}_{1100} ) = - ln ( \frac{7}{15} ) = 0.76$$

and phenotype 1110 has

$$IC ( {c}_{1110} ) = - ln ( \frac{3}{15} ) = 1.61 .$$

Further, the common ancestors shared between phenotype 1100 and phenotype 1110 include phenotype 1100 and phenotype 1000. Since phenotype 1100 has a greater information content (i.e., IC(c₁₁₀₀)=0.76) than phenotype 1000 (i.e., IC(c₁₀₀₀)=0), the maximum information content of any common ancestor shared between phenotype 1110 and phenotype 1100 is the information content of phenotype 1100, which is 0.76. Lastly, to calculate the overall similarity score (e.g., the Lin similarity score) between phenotype 1100 and 1110, the following formula is used:

$${sim}_{Lin} ( {c}_{1} , {c}_{2} ) = \frac{2 \times MICA ( {c}_{1} , {c}_{2} )}{IC ( {c}_{1} ) + IC ( {c}_{2} )}$$

where sim_{Lin}(c₁,c₂) is the overall similarity score (i.e., the Lin similarity) and MICA(c₁,c₂) is the maximum information content of any common ancestor. Hence, using the phenotype 1100 and phenotype 1110 example associated with FIG. 7, the overall similarity score would be:

$${sim}_{Lin} ( {c}_{1100} , {c}_{1110} ) = \frac{2 \times 0.76}{0.76 + 1.61} = 0.64$$

The similarity calculation provided above could be performed for every pair of phenotypes within the ontology. Likewise, for step 608 of the process 600 of FIG. 6, a similarity calculation could be performed for every possible pair of phenotypes that includes both a phenotype from the first data set and a phenotype from the second data set.

An alternative technique for calculating similarity scores could also be used. For example, the Resnik similarity may be used to determine similarity scores. Resnik similarity between two phenotypes within an ontology may be defined as:

$${sim}_{Res} ( {c}_{1} , {c}_{2} ) = \frac{MICA ( {c}_{1} , {c}_{2} )}{MICO} ,$$

$$if$$

$${c}_{1} \neq {c}_{2}$$

$${sim}_{Res} ( {c}_{1} , {c}_{2} ) = 1 ,$$

$$if$$

$${c}_{1} = {c}_{2}$$

where sim_{Res}(c₁,c₂) is the overall similarity score (i.e., the Resnik similarity), MICA(c₁,c₂) is the maximum information content of any common ancestor, and MICO is the maximum information content in the entire ontology. Information content is calculated according to the same formula as listed above for Lin similarity.

Returning to the example of phenotype 1100 and phenotype 1110, the maximum information content in the entire ontology would be the information content of any of phenotypes 1111, 1112, 1121, 1122, 1211, 1212, 1221, or 1222, which is, for example,

$$IC ( {c}_{1111} ) = - ln ( \frac{1}{15} ) = 2.71$$

Further, the overall Resnik similarity between those two phenotypes would be, since c₁₁₀₀≠c₁₁₁₀:

$${sim}_{Res} ( {c}_{1100} , {c}_{1110} ) = \frac{MICA ( {c}_{1100} , {c}_{1110} )}{MICO} = \frac{0.76}{2.71} = 0.28$$

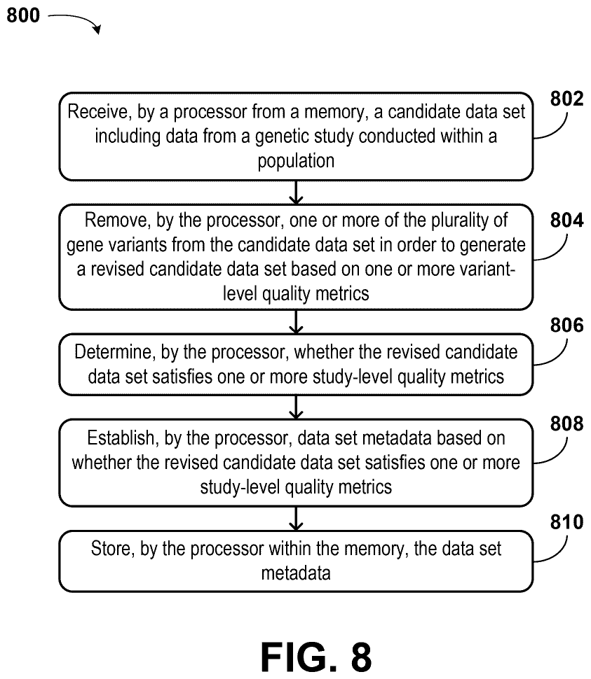

FIG. 8 is a flowchart diagram of a method 800, according to example embodiments. In some embodiments, the method 800 may be performed by a computing system (e.g., the computing system 100 shown and described with reference to FIGS. 1 and 2). In some embodiments, the method 800 may include performing a quality assessment.

At block 802, the method 800 may include receiving, by a processor (e.g., the processor 102) from a memory (e.g., the memory 110, the removable mass storage device 112, or the fixed mass storage device 120), a candidate data set including data from a genetic study conducted within a population. The data from the genetic study may include a plurality of gene variants determined within the population.

At block 804, the method 800 may include removing, by the processor, one or more of the plurality of gene variants from the candidate data set in order to generate a revised candidate data set based on one or more variant-level quality metrics.

At block 806, the method 800 may include determining, by the processor, whether the revised candidate data set satisfies one or more study-level quality metrics.

At block 808, the method 800 may include establishing, by the processor, data set metadata based on whether the revised candidate data set satisfies one or more study-level quality metrics.

At block 810, the method 800 may include storing, by the processor within the memory, the data set metadata.

In some embodiments, the method 800 may also include recalculating, by the processor after removing the one or more gene variants from the candidate data set, one or more statistics within the revised candidate data set based on the plurality of gene variants within the revised candidate data set.

In some embodiments of the method 800, the one or more study-level quality metrics may include a minimum threshold number of unique gene variants within the genetic study.

In some embodiments of the method 800, the minimum threshold number of unique gene variants may be 1 million or more.

In some embodiments of the method 800, the one or more study-level quality metrics may include a minimum sample size within the genetic study.

In some embodiments of the method 800, the one or more variant-level quality metrics may include a minimum threshold number of occurrences of a given gene variant within the genetic study.

In some embodiments of the method 800, block 804 may include determining, by the processor for each gene variant within the genetic study, a minimum threshold number of occurrences of the respective gene variant. Block 804 may also include removing, by the processor, the respective gene variant from the candidate data set if the respective gene variant does not occur at least the minimum threshold number of occurrences within the candidate data set.

In some embodiments of the method 800, determining the minimum threshold number of occurrences of the respective gene variant may include receiving, by the processor, an alternative data set including data from an alternative genetic study conducted within an alternative population. Determining the minimum threshold number of occurrences may also include matching, by the processor, the respective gene variant to a corresponding gene variant within the alternative data set. The corresponding gene variant may be a gene variant within the alternative data set that is maximally analogous to the corresponding gene variant. Additionally, determining the minimum threshold number of occurrences may include determining, by the processor, a minor allele frequency of the alternative gene variant within the alternative data set based on a number of occurrences of the alternative gene variant within the alternative data set. Further, determining the minimum threshold number of occurrences may include determining, by the processor, the minimum threshold number of occurrences based on the minor allele frequency.

In some embodiments of the method 800, determining the minimum threshold number of occurrences based on the minor allele frequency may include setting the minimum threshold number of occurrences equal to two times the minor allele frequency times a number of individuals within the population.

In some embodiments of the method 800, the minimum threshold number of occurrences may be 5 or more.

In some embodiments of the method 800, block 804 may include determining, by the processor for each gene variant within the genetic study according to a variant taxonomy, a variant identifier that characterizes a variant type for the respective gene variant. Block 804 may also include removing, by the processor, the respective gene variant from the candidate data set if: the variant identifier for the respective gene variant matches a variant identifier determined for a different gene variant within the genetic study; or the variant identifier is missing one or more pieces of information defined by the variant taxonomy.

In some embodiments of the method 800, the variant taxonomy may include a chromosome, position, reference allele, and alternative allele (CPRA) identification relative to the Genome Research Consortium human build 38 (GRCh38).

In some embodiments of the method 800, block 806 may include determining, by the processor, a proportion of gene variants within the genetic study that have either: a variant identifier that matches a variant identifier of a different gene variant within the genetic study; or a variant identifier that is missing one or more pieces of information defined by the variant taxonomy. Block 806 may also include comparing, by the processor, the proportion of gene variants to a maximum threshold value.

In some embodiments of the method 800, the maximum threshold value may be 0.1 or less.

In some embodiments of the method 800, block 806 may include determining, by the processor, whether the data in the revised candidate data set includes whole-exome data.

In some embodiments of the method 800, block 806 may include determining, by the processor, whether a genetic study of the revised candidate data set is a study of a quantitative trait.

In some embodiments of the method 800, block 806 may include removing, by the processor, a gene variant associated with a chromosome other than chromosomes 1-22 and the X chromosome. Additionally or alternatively, block 806 may include removing, by the processor, a gene variant that lacks a complete set of variant metadata within the candidate data set. In some embodiments, block 806 may include removing, by the processor, a gene variant that is identical to another gene variant within the candidate data set. In some embodiments, block 806 may include removing, by the processor, a gene variant that has an associated p-value within the candidate data set that is outside of a range from 0 to 1, inclusive. In some embodiments, block 806 may include removing, by the processor, a gene variant that has an associated error within the candidate data set that is less than or equal to 0.

In some embodiments of the method 800, the genetic study may be a case-control study. Further, block 806 may include determining, by the processor, a logarithm of an odds ratio for each gene variant in the revised candidate data set. Block 806 may also include determining, by the processor, a first quartile from among the logarithms of odds ratios for the gene variants in the revised candidate data set. Additionally, block 806 may include comparing, by the processor, the first quartile to a first threshold value. Still further, block 806 may include determining, by the processor, a third quartile from among the logarithms of odds ratios for the gene variants in the revised candidate data set. In addition, block 806 may include comparing, by the processor, the third quartile to a second threshold value. Yet further, block 806 may include determining, by the processor, that the revised candidate data set fails to satisfy the one or more study-level quality metrics if: the first quartile is less than the first threshold value; or the third quartile is greater than the second threshold value.

In some embodiments of the method 800, the first threshold value may be −0.2 or less. Additionally, the second threshold value may be 0.2 or more.

In some embodiments of the method 800, block 806 may include performing, by the processor, a chi-squared test for each of the gene variants in the revised candidate data set to determine a chi-squared value for each of the gene variants. Block 806 may also include determining, by the processor, a median chi-squared value from among the chi-squared values. Additionally, block 806 may include determining, by the processor, a genomic inflation factor by dividing the median chi-squared value by an expected median of a chi-squared distribution with an appropriate corresponding number of degrees of freedom. Further, block 806 may include comparing, by the processor, the genomic inflation factor to a minimum threshold genomic inflation factor. In addition, block 806 may include comparing, by the processor, the genomic inflation factor to a maximum threshold genomic inflation factor. Yet further, block 806 may include determining, by the processor, that the revised candidate data set does not satisfy the one or more study-level quality metrics if the genomic inflation factor is less than the minimum threshold genomic inflation factor or greater than the maximum threshold genomic inflation factor.

In some embodiments of the method 800, the minimum threshold genomic inflation factor may be 0.9 or more and the maximum threshold genomic inflation factor may be 1.5 or less.

In some embodiments of the method 800, block 806 may include performing, by the processor, a chi-squared test for each of the gene variants in the revised candidate data set to determine a chi-squared value for each of the gene variants. Block 806 may also include determining, by the processor, a median chi-squared value from among the chi-squared values. Additionally, block 806 may include determining, by the processor, a genomic inflation factor by dividing the median chi-squared value by an expected median of a chi-squared distribution with an appropriate corresponding number of degrees of freedom. Further, block 806 may include normalizing, by the processor, the genomic inflation factor to a study having 1000 cases and 1000 controls to determine a normalized genomic inflation factor. In addition, block 806 may include comparing, by the processor, the normalized genomic inflation factor to a maximum threshold normalized genomic inflation factor. Still further, block 806 may include determining, by the processor, that the revised candidate data set does not satisfy the one or more study-level quality metrics if the normalized genomic inflation factor is greater than the maximum threshold normalized genomic inflation factor.

In some embodiments of the method 800, the maximum threshold normalized genomic inflation factor may be 1.5 or less.

In some embodiments of the method 800, block 806 may include determining, by the processor, a minor allele frequency for each gene variant within the revised candidate data set based on a number of occurrences. Block 806 may also include separating, by the processor, each of the gene variants into a plurality of frequency bins based on the minor allele frequency associated with the gene variants. Additionally, block 806 may include, for each frequency bin, performing, by the processor, a chi-squared test for each of the gene variants in the frequency bin relative to the other gene variants in the frequency bin to determine a chi-squared value for each of the gene variants in the bin. Further, block 806 may include, for each frequency bin, determining, by the processor, a median chi-squared value from among the chi-squared values. In addition, block 806 may include, for each frequency bin, determining, by the processor, a genomic inflation factor by dividing the median chi-squared value by an expected median of a chi-squared distribution with an appropriate corresponding number of degrees of freedom. Still further, block 806 may include, for each frequency bin, normalizing, by the processor, the genomic inflation factor to a study having a predetermined number of cases and a predetermined number of controls to determine a normalized genomic inflation factor. Yet further, block 806 may include dividing, by the processor, the normalized genomic inflation factor having the highest value from among the frequency bins with the normalized genomic inflation factor having the lowest value from among the frequency bins to determine a normalized genomic inflation factor ratio. Even further, block 806 may include comparing, by the processor, the normalized genomic inflation factor ratio to a maximum threshold normalized genomic inflation factor ratio. Still yet further, block 806 may include determining, by the processor, that the revised candidate data set does not satisfy the one or more study-level quality metrics if the normalized genomic inflation factor ratio is greater than the maximum threshold normalized genomic inflation factor ratio.

In some embodiments of the method 800, the plurality of frequency bins may include two frequency bins, three frequency bins, four frequency bins, five frequency bins, six frequency bins, seven frequency bins, eight frequency bins, nine frequency bins, ten frequency bins, eleven frequency bins, twelve frequency bins, thirteen frequency bins, fourteen frequency bins, or fifteen frequency bins.

In some embodiments of the method 800, the maximum threshold normalized genomic inflation factor ratio is 2.0 or less.

In some embodiments of the method 800, the candidate data set may include data from a GWAS conducted within the population.

In some embodiments, the method 800 may also include storing, by the processor within the memory or an auxiliary memory, the revised candidate data set when the revised candidate data set satisfies the one or more study-level quality metrics.

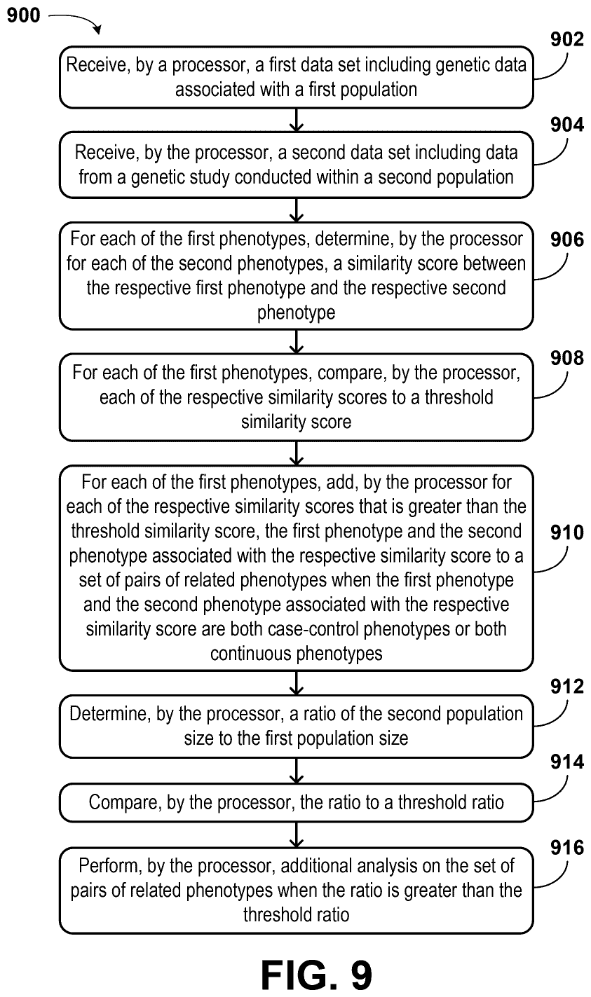

FIG. 9 is a flowchart diagram of a method 900, according to example embodiments. In some embodiments, the method 900 may be performed by a computing system (e.g., the computing system 100 shown and described with reference to FIGS. 1 and 2). In some embodiments, the method 900 may include performing a merging technique.

At block 902, the method 900 may include receiving, by a processor, a first data set including genetic data associated with a first population. The genetic data may include a plurality of first phenotypes associated with a plurality of first gene variants determined within the first population.

At block 904, the method 900 may include receiving, by the processor, a second data set including data from a genetic study conducted within a second population. The data from the genetic study may include a plurality of second phenotypes associated with a plurality of second gene variants determined within the second population.

At block 906, the method 900 may include, for each of the first phenotypes, determining, by the processor for each of the second phenotypes, a similarity score between the respective first phenotype and the respective second phenotype. Determining the similarity score may include comparing the first phenotype to the second phenotype using an ontology.

At block 908, the method 900 may include, for each of the first phenotypes, comparing, by the processor, each of the respective similarity scores to a threshold similarity score.

At block 910, the method 900 may include, for each of the first phenotypes, adding, by the processor for each of the respective similarity scores that is greater than the threshold similarity score, the first phenotype and the second phenotype associated with the respective similarity score to a set of pairs of related phenotypes when the first phenotype and the second phenotype associated with the respective similarity score are both case-control phenotypes or both continuous phenotypes.

At block 912, the method 900 may include determining, by the processor, a ratio of the second population size to the first population size.

At block 914, the method 900 may include comparing, by the processor, the ratio to a threshold ratio.

At block 916, the method 900 may include performing, by the processor, additional analysis on the set of pairs of related phenotypes when the ratio is greater than the threshold ratio.

In some embodiments of the method 900, the ontology may include the MeSH ontology or the EFO.

In some embodiments of the method 900, the threshold similarity score may be at least 0.95.

In some embodiments of the method 900, the threshold ratio may be 0.1 or more.

In some embodiments of the method 900, block 916 may include, for each pair of related phenotypes within the set, determining, by the processor, a first description of the respective first phenotype based on data set metadata associated with the first data set. Block 916 may also include, for each pair of related phenotypes within the set, determining, by the processor, a second description of the respective second phenotype based on data set metadata associated with the second data set. Additionally, block 916 may include, for each pair of related phenotypes within the set, comparing, by the processor, the first description to the second description to determine whether to perform further analysis.

In some embodiments of the method 900, comparing the first description to the second description may include determining whether the first description and the second description share a threshold extent of similarity.

In some embodiments of the method 900, comparing the first description to the second description may include determining whether phenotypes of the first description represent a subset or a superset of phenotypes of the second description.

In some embodiments of the method 900, block 916 may include determining, by the processor based on data set metadata associated with the first data set and data set metadata associated with the second data set, a number representing how many individuals within the second population are not present within the first population. Block 916 may also include comparing, by the processor, the number to a threshold uniqueness value to determine whether to perform further analysis.

In some embodiments of the method 900, block 906 includes associating, by the processor, a first tag with the first phenotype. Further, the first tag for the first phenotype may have been determined based on a first user input.

In some embodiments of the method 900, block 906 includes associating, by the processor, a first tag with the first phenotype. The first tag for the first phenotype may have been determined by retrieving a list of published tags with an associated list of descriptions. The first tag for the first phenotype may also have been determined by associating a description of the first phenotype with a description on the associated list of descriptions.

In some embodiments of the method 900, block 906 includes associating, by the processor, a first tag with the first phenotype. The first tag for the first phenotype may have been determined by applying mapping techniques in a sequential fashion from a most-effective technique to a least-effective technique until a tag with a threshold mapping score was identified. Additionally, each of the mapping techniques may be separately usable to determine a tag associated with a phenotype.

In some embodiments of the method 900, block 906 may include associating, by the processor, a first tag with the first phenotype. The first tag for the first phenotype may have been determined using an ontological mapping technique. The ontological mapping technique may include performing a TF-IDF calculation or a string distance calculation to determine a mapping score. The ontological mapping technique may also include comparing the mapping score to a threshold mapping score.

In some embodiments of the method 900, block 906 may include associating, by the processor, a first tag with the first phenotype. The first tag for the first phenotype may have been determined by matching a first string of text of a descriptor associated with the phenotype to a second string of text of a descriptor associated with a label or an alias defined within the ontology.

In some embodiments of the method 900, block 916 may include identifying, by the processor, a locus associated with a locus phenotype. Block 916 may also include determining, by the processor, that multiple gene variants are associated independently with the locus phenotype. Determining that multiple gene variants are associated independently with the locus phenotype may include applying, by the processor, conditional and joint association analysis using data set metadata associated with the second data set. The data set metadata associated with the second data set may include summary statistics and a linkage disequilibrium reference panel.

In some embodiments of the method 900, the second data set may include data from a GWAS conducted within the second population.

### IV. CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including RAM, a disk drive, a solid state drive, or another storage medium.

The computer-readable medium can also include non-transitory computer-readable media such as computer-readable media that store data for short periods of time like register memory and processor cache. The computer-readable media can further include non-transitory computer-readable media that store program code and/or data for longer periods of time. Thus, the computer-readable media may include secondary or persistent long term storage, like ROM, optical or magnetic disks, solid state drives, CD-ROM, for example. The computer-readable media can also be any other volatile or non-volatile storage systems. A computer-readable medium can be considered a computer-readable storage medium, for example, or a tangible storage device.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A method comprising:
receiving, by a processor from a memory, a candidate data set comprising data from a genetic study conducted within a population, wherein the data from the genetic study comprises a plurality of gene variants determined within the population;
removing, by the processor, one or more of the plurality of gene variants from the candidate data set in order to generate a revised candidate data set based on one or more variant-level quality metrics;
determining, by the processor, whether the revised candidate data set satisfies one or more study-level quality metrics;
establishing, by the processor, data set metadata based on whether the revised candidate data set satisfies one or more study-level quality metrics; and
storing, by the processor within the memory, the data set metadata.

2. The method of claim 1, further comprising recalculating, by the processor after removing the one or more gene variants from the candidate data set, one or more statistics within the revised candidate data set based on the plurality of gene variants within the revised candidate data set.

3. The method of claim 1, wherein the one or more study-level quality metrics comprise a minimum threshold number of unique gene variants within the genetic study.

4. The method of claim 1, wherein the one or more study-level quality metrics comprise a minimum sample size within the genetic study or a minimum threshold number of occurrences of a given gene variant within the genetic study.

5. The method of claim 1, wherein removing, by the processor, the one or more gene variants from the candidate data set in order to generate the revised candidate data set based on one or more variant-level quality metrics comprises:
determining, by the processor for each gene variant within the genetic study, a minimum threshold number of occurrences of the respective gene variant; and
removing, by the processor, the respective gene variant from the candidate data set if the respective gene variant does not occur at least the minimum threshold number of occurrences within the candidate data set.

6. The method of claim 5, wherein determining the minimum threshold number of occurrences of the respective gene variant comprises:
receiving, by the processor, an alternative data set comprising data from an alternative genetic study conducted within an alternative population;
matching, by the processor, the respective gene variant to a corresponding gene variant within the alternative data set, wherein the corresponding gene variant is a gene variant within the alternative data set that is maximally analogous to the corresponding gene variant;
determining, by the processor, a minor allele frequency of the alternative gene variant within the alternative data set based on a number of occurrences of the alternative gene variant within the alternative data set; and
determining, by the processor, the minimum threshold number of occurrences based on the minor allele frequency.

7. The method of claim 6, wherein determining the minimum threshold number of occurrences based on the minor allele frequency comprises setting the minimum threshold number of occurrences equal to two times the minor allele frequency times a number of individuals within the population.

8. The method of claim 1, wherein removing, by the processor, the one or more gene variants from the candidate data set in order to generate the revised candidate data set based on one or more variant-level quality metrics comprises:
determining, by the processor for each gene variant within the genetic study according to a variant taxonomy, a variant identifier that characterizes a variant type for the respective gene variant; and
removing, by the processor, the respective gene variant from the candidate data set if:
the variant identifier for the respective gene variant matches a variant identifier determined for a different gene variant within the genetic study; or
the variant identifier is missing one or more pieces of information defined by the variant taxonomy.

9. The method of claim 8, wherein the variant taxonomy comprises a chromosome, position, reference allele, and alternative allele (CPRA) identification relative to the Genome Research Consortium human build 38 (GRCh38).

10. The method of claim 8, wherein determining whether the revised candidate data set satisfies one or more study-level quality metrics comprises:
determining, by the processor, a proportion of gene variants within the genetic study that have either:
a variant identifier that matches a variant identifier of a different gene variant within the genetic study; or
a variant identifier that is missing one or more pieces of information defined by the variant taxonomy; and
comparing, by the processor, the proportion of gene variants to a maximum threshold value.

11. The method of claim 1, wherein determining, by the processor, whether the revised candidate data set satisfies one or more study-level quality metrics comprises:
determining, by the processor, whether the data in the revised candidate data set comprises whole-exome data; or
determining, by the processor, whether a genetic study of the revised candidate data set is a study of a quantitative trait.

12. The method of claim 1, wherein removing, by the processor, the one or more gene variants from the candidate data set in order to generate the revised candidate data set based on one or more variant-level quality metrics comprises:
removing, by the processor, a gene variant associated with a chromosome other than chromosomes 1-22 and the X chromosome;
removing, by the processor, a gene variant that lacks a complete set of variant metadata within the candidate data set;
removing, by the processor, a gene variant that is identical to another gene variant within the candidate data set;
removing, by the processor, a gene variant that has an associated p-value within the candidate data set that is outside of a range from 0 to 1, inclusive; or
removing, by the processor, a gene variant that has an associated error within the candidate data set that is less than or equal to 0.

13. The method of claim 1, wherein the genetic study is a case-control study, and wherein determining, by the processor, whether the revised candidate data set satisfies the one or more study-level quality metrics comprises:
determining, by the processor, a logarithm of an odds ratio for each gene variant in the revised candidate data set;
determining, by the processor, a first quartile from among the logarithms of odds ratios for the gene variants in the revised candidate data set;
comparing, by the processor, the first quartile to a first threshold value;
determining, by the processor, a third quartile from among the logarithms of odds ratios for the gene variants in the revised candidate data set;
comparing, by the processor, the third quartile to a second threshold value; and
determining, by the processor, that the revised candidate data set fails to satisfy the one or more study-level quality metrics if:
the first quartile is less than the first threshold value; or
the third quartile is greater than the second threshold value.

14. The method of claim 1, wherein determining, by the processor, whether the revised candidate data set satisfies the one or more study-level quality metrics comprises:
performing, by the processor, a chi-squared test for each of the gene variants in the revised candidate data set to determine a chi-squared value for each of the gene variants;
determining, by the processor, a median chi-squared value from among the chi-squared values;
determining, by the processor, a genomic inflation factor by dividing the median chi-squared value by an expected median of a chi-squared distribution with an appropriate corresponding number of degrees of freedom;
comparing, by the processor, the genomic inflation factor to a minimum threshold genomic inflation factor;
comparing, by the processor, the genomic inflation factor to a maximum threshold genomic inflation factor; and
determining, by the processor, that the revised candidate data set does not satisfy the one or more study-level quality metrics if the genomic inflation factor is less than the minimum threshold genomic inflation factor or greater than the maximum threshold genomic inflation factor.

15. The method of claim 1, wherein determining, by the processor, whether the revised candidate data set satisfies the one or more study-level quality metrics comprises:
performing, by the processor, a chi-squared test for each of the gene variants in the revised candidate data set to determine a chi-squared value for each of the gene variants;
determining, by the processor, a median chi-squared value from among the chi-squared values;
determining, by the processor, a genomic inflation factor by dividing the median chi-squared value by an expected median of a chi-squared distribution with an appropriate corresponding number of degrees of freedom;
normalizing, by the processor, the genomic inflation factor to a study having 1000 cases and 1000 controls to determine a normalized genomic inflation factor;
comparing, by the processor, the normalized genomic inflation factor to a maximum threshold normalized genomic inflation factor; and
determining, by the processor, that the revised candidate data set does not satisfy the one or more study-level quality metrics if the normalized genomic inflation factor is greater than the maximum threshold normalized genomic inflation factor.

16. The method of claim 1, wherein determining, by the processor, whether the revised candidate data set satisfies the one or more study-level quality metrics comprises:
determining, by the processor, a minor allele frequency for each gene variant within the revised candidate data set based on a number of occurrences;
separating, by the processor, each of the gene variants into a plurality of frequency bins based on the minor allele frequency associated with the gene variants;
for each frequency bin:
performing, by the processor, a chi-squared test for each of the gene variants in the frequency bin relative to the other gene variants in the frequency bin to determine a chi-squared value for each of the gene variants in the bin;
determining, by the processor, a median chi-squared value from among the chi-squared values;
determining, by the processor, a genomic inflation factor by dividing the median chi-squared value by an expected median of a chi-squared distribution with an appropriate corresponding number of degrees of freedom; and
normalizing, by the processor, the genomic inflation factor to a study having a predetermined number of cases and a predetermined number of controls to determine a normalized genomic inflation factor;
dividing, by the processor, the normalized genomic inflation factor having the highest value from among the frequency bins with the normalized genomic inflation factor having the lowest value from among the frequency bins to determine a normalized genomic inflation factor ratio;
comparing, by the processor, the normalized genomic inflation factor ratio to a maximum threshold normalized genomic inflation factor ratio; and
determining, by the processor, that the revised candidate data set does not satisfy the one or more study-level quality metrics if the normalized genomic inflation factor ratio is greater than the maximum threshold normalized genomic inflation factor ratio.

17. The method of claim 1, wherein the candidate data set comprises data from a genome-wide association study (GWAS) conducted within the population.

18. The method of claim 1, further comprising storing, by the processor within the memory or an auxiliary memory, the revised candidate data set when the revised candidate data set satisfies the one or more study-level quality metrics.

19. A non-transitory, computer-readable medium having instructions stored thereon, wherein the instructions, when executed by a processor, cause the processor to:
receive, from a memory, a candidate data set comprising data from a genetic study conducted within a population, wherein the data from the genetic study comprises a plurality of gene variants determined within the population;
remove one or more of the plurality of gene variants from the candidate data set in order to generate a revised candidate data set based on one or more variant-level quality metrics;
determine whether the revised candidate data set satisfies one or more study-level quality metrics;
establish data set metadata based on whether the revised candidate data set satisfies one or more study-level quality metrics; and
store, within the memory, the data set metadata.

20. A system comprising one or more processors configured to:
receive, from a memory, a candidate data set comprising data from a genetic study conducted within a population, wherein the data from the genetic study comprises a plurality of gene variants determined within the population;
remove one or more of the plurality of gene variants from the candidate data set in order to generate a revised candidate data set based on one or more variant-level quality metrics;
determine whether the revised candidate data set satisfies one or more study-level quality metrics;
establish data set metadata based on whether the revised candidate data set satisfies one or more study-level quality metrics; and
store, within the memory, the data set metadata.
